(19) United States
(12) Patent Application Publication
(10) Pub. No.: US 2013/0164367 A1
(43) Pub. Date: Jun. 27, 2013

(54) TREATMENT OF NEURODEGENERATIVE DISEASE WITH CREB-BINDING PROTEIN

(71) Applicant: The Board of Regents of the University of Texas S, Austin, TX (US)

(72) Inventors: Salvatore Oddo, San Antonio, TX (US); Antonella Caccamo, San Antonio, TX (US)

(73) Assignee: The Board of Regents of the University of Texas System, Austin, TX (US)

(21) Appl. No.: 13/708,444

(22) Filed: Dec. 7, 2012

Related U.S. Application Data
(60) Provisional application No. 61/568,458, filed on Dec. 8, 2011.

(51) Int. Cl. A61K 38/02

(52) U.S. Cl. CPC A61K 38/02
USPC 424/450; 514/8.3; 514/44 R

(57) ABSTRACT

The present invention relates to improving cognition and treating of neurodegenerative disease using CREB-binding protein. The protein may be delivered to the brain by an expression vector, in particular a lentiviral vector.

This application claims benefit of priority to U.S. Provisional Application Ser. No. 61/568,458, filed Dec. 8, 2011, the entire contents of which are hereby incorporated by reference.

This invention was made with government support under grant no. 1R00AG029729 awarded by the National Institutes of Health. The government has certain rights in the invention.

### BACKGROUND OF THE INVENTION

I. Field of the Invention

The present invention relates generally to the fields of medicine, neuropathology and molecular biology. More particularly, it concerns the use or expression of CREB binding protein as a therapeutic agent for the treatment of neurodegenerative diseases such as Alzheimer's Disease.

II. Description of Related Art

Neurodegenerative diseases are generally characterized by the loss of neurons from one or more regions of the central nervous system. They are complex in both origin and progression, and have proved to be some of the most difficult types of disease to treat. In fact, for some neurodegenerative diseases, there are no drugs available that provide significant therapeutic benefit. The difficulty in providing therapy is all the more tragic given the devastating effects these diseases have on their victims.

One type of neurodegenerative disease is Alzheimer's Disease (AD), the most common form of dementia among older people. Scientists believe that up to 4 million Americans suffer from AD. The disease usually begins after age 60, and risk goes up with age. While younger people also may get AD, it is much less common. About 3 percent of men and women ages 65 to 74 have AD, and nearly half of those age 85 and older may have the disease. While the subject of intensive research, the precise causes of AD are still unknown, and there is no cure.

AD attacks parts of the brain that control thought, memory, and language. Abnormal clumps, now called amyloid “plaques,” and tangled bundles of fibers, now called neurofibrillary “tangles,” are considered hallmarks of AD. The production, aggregation, and accumulation of amyloid β-protein (Aβ), the major constituent of the amyloid plaque, in the brain are initial steps in the pathogenesis of AD. Aβ is generated by the intracellular processing of amyloid β precursor protein (APP, see FIG. 1) (Selkoe, 2001), a type I membrane protein (Kang et al., 1987), by proteases β-secretase (memapsin 2 or BACE1) and γ-secretase. The cytoplasmic domain of APP (APPcyt), through its interactions with cytoplasmic proteins, plays an important role in the regulation of APP metabolism and Aβ production (King and Turner, 2004).

NMDA receptors are fundamental for synaptic plasticity and long-term potentiation, and in this context, it has been shown that Aβ accumulation also reduces glutamatergic transmission and inhibits synaptic plasticity by interfering with NMDA receptor endocytosis, thereby reducing their availability at synapses (Snyder et al., 2005; Palop and Mucke, 2010). These results are consistent with a reduction in expression levels of proteins playing an important role in synaptic plasticity, such as NR2B and GluR1, in transgenic mice (Dickey et al., 2003). Furthermore, Aβ accumulation is shown to alter other transduction pathways involved in learning and memory (Snyder at al., 2005; Caccamo at al., 2010; Ma et al., 2007; Palop et al., 2005; Palop et al., 2003).

The critical role of immediate early genes (IEGs) in memory formation is widely accepted (Lanahan and Worley, 1998). The expression of some of these IEGs is reduced in AD, as shown by in vitro and in vivo experiments (Dickey at al., 2003; Ma et al., 2007; Palop et al., 2005; Palop et al., 2003; Vitolo at al., 2002; Tong at al., 2001). Most recently, it was reported that gene transcription, mediated by the cAMP-response element binding protein (CREB)-regulated transcription coactivator CRTC1, is impaired in a mouse model of AD (Espana at al., 2010), further suggesting that Aβ-induced memory deficits maybe due to alterations in signaling transduction pathways. CREB is a key immediate early gene involved in learning and memory. The CREB-binding protein (CBP) is a transcriptional coactivator whose function is critical for CREB activity and learning and memory (Goodman and Smolik, 2000). Structurally, CBP has several protein-binding regions and a histone acetyltransferase (HAT) domain; functionally, CBP acts as a transcriptional coactivator by facilitating the recruitment of required components of the transcriptional machinery, and as a FIAT by altering chromatin structure (Vo and Goodman, 2001). However, the actual role of CBP in AD is unclear, and contradicting reports have been published (Francis et al 2006; Marambaud et al., 2003; Saura et al., 2004).

### SUMMARY OF THE INVENTION

Thus, in accordance with the present invention, there is provided a method of increasing brain-derived neurotrophic factor (BDNF) in the brain of a subject comprising providing to the subject a CREB-binding protein (CBP). The CBP may be provided by administration of an expression vector to the subject. The administration may comprise injection or infusion using stereotactic surgical techniques. The expression vector may be a viral vector, such as neutrophic viral vector, such as a retroviral vector, a lentiviral vector, a herpesviral vector, an adenoviral vector or an adeno-associated viral vector. The expression vector may be a non-viral vector, such as one contained in a lipid delivery vehicle or nanoparticle. The lipid delivery vehicle may be a liposome. Providing may comprise, daily, every other day, every third day, every fourth day, every fifth day, every sixth day or weekly administration. The subject may or may not have been diagnosed with neurodegenerative diseases such as Alzheimer's Disease (AD), Huntington's Disease (HD), Rubinstein-Taybe syndrome (RTS) and amyotrophic lateral sclerosis (ALS). The subject may have a familial history of neurodegenerative diseases such as Alzheimer's Disease. The method may further comprise treating the subject with a second neurodegenerative diseasetherapy, such as a cognitive therapy.

In another embodiment, there is provided a method of improving learning and/or reducing memory deficits in a subject comprising providing to the subject CREB-binding protein (CBP). The CBP may be provided by administration of an expression vector to the subject. The administration may comprise injection or infusion using stereotactic surgical techniques. The expression vector may be a viral vector, such as neutrophic viral vector, such as a retroviral vector, a lentiviral vector, a herpesviral vector, an adenoviral vector or an adeno-associated viral vector. The expression vector may be a non-viral vector, such as one contained in a lipid delivery vehicle or nanoparticle. The lipid delivery vehicle may be a liposome. Providing may comprise, daily, every other day, every third day, every fourth day, every fifth day, every sixth day or weekly administration. The subject may or may not have been diagnosed with neurodegenerative diseases such as Alzheimer's Disease (AD), Huntington's Disease (HD), Rubinstein-Taybe syndrome (RTS) and amyotrophic lateral sclerosis (ALS). The subject may have a familial history of neurodegenerative diseases such as Alzheimer's Disease (AD), Huntington's Disease (HD), Rubinstein-Taybe syndrome (RTS) and amyotrophic lateral sclerosis (ALS). The method may further comprise treating the subject with a second neurodegenerative disease therapy, such as a cognitive therapy.

In yet another embodiment, there is provided a method of treating a neurodegenerative disease in a subject comprising providing to the subject CREB-binding protein (CBP). The CBP may be provided by administration of an expression vector to the subject. The administration may comprise injection or infusion using stereotactic surgical techniques. The expression vector may be a viral vector, such as neutrophic viral vector, such as a retroviral vector, a lentiviral vector, a herpesviral vector, an adenoviral vector or an adeno-associated viral vector. The expression vector may be a non-viral vector, such as one contained in a lipid delivery vehicle or nanoparticle. The lipid delivery vehicle may be a liposome. Providing may comprise, daily, every other day, every third day, every fourth day, every fifth day, every sixth day or weekly administration. The subject may or may not have been diagnosed with neurodegenerative diseases such as Alzheimer's Disease (AD), Huntington's Disease (HD), Rubinstein-Taybe syndrome (RTS) and amyotrophic lateral sclerosis (ALS). The subject may have a familial history of neurodegenerative diseases such as Alzheimer's Disease (AD), Huntington's Disease (HD), Rubinstein-Taybe syndrome (RTS) and amyotrophic lateral sclerosis (ALS). The method may further comprise treating the subject with a second neurodegenerative disease therapy, such as a cognitive therapy.

In still yet another embodiment, there is provided a pharmaceutical composition comprising an expression construct comprising a promoter active in neuronal cells operably connected to a nucleic acid segment coding for a CREB-binding protein (CBP) disposed in a pharmaceutically acceptable carrier, diluent or buffer. The promoter may be EF1a, alpha-synuclein, or beta-actin. The expression construct may be a neurotrophic viral expression construct, such as a lentiviral, retroviral, herpesviral, adenoviral or an adeno-associated viral expression construct. The expression construct may be a non-viral expression construct conjugated to or entrapped within a delivery particle.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### BRIEF DESCRIPTION OF THE DRAWINGS

The following drawings form part of the present specification and are included to further demonstrate certain aspects of the present invention.

FIGS. 1A-D. Activity-dependent CREB activation is impaired in the 3×Tg-AD mice. (FIG. 1A) Learning abilities of 6-month-old 3×Tg-AD and Non-Tg mice (n=16/genotype) were evaluated in the spatial reference version of the Morris water maze (MWM). At the end of the third day, 8 mice per genotype were sacrificed and the remaining mice were trained for two additional days. At day 4 and 5 of training, the Non-Tg mice performed significantly better than the 3×Tg-AD mice as denoted by a shorter time to find the hidden platform. (FIG. 1B) Representative Western blots (probed with the indicated antibodies) of proteins extracted from the hippocampi of mice sacrificed directly from their home cages or trained in MWM for 3 or 5 days. (FIGS. 1C-D) Quantitative analyses of the blots indicate that total CREB levels were similar between Non-Tg and 3×Tg-AD mice at baseline and after neuronal stimulation. In contrast, at baseline, the levels of phosphorylated CREB at Ser133 (pCREB) were significantly reduced in the brains of the 3×Tg-AD compared to Non-Tg mice. Although the percentage increase in pCREB levels in the Non-Tg mice was not statistically different from that of the 3×Tg-AD mice, the absolute levels of pCREB were significantly lower in the 3×Tg-AD mice at all the time-points analyzed. Protein levels are expressed as fold changes over sham-injected Non-Tg mice and represent means±SEM. * indicates p<0.05; ** indicates p<0.01.

FIGS. 2A-B. NMDA signaling is impaired in the 3×Tg-AD mice. (FIG. 2A) Representative Western blots of proteins extracted from the hippocampi of 6-month-old 3×Tg-AD and Non-Tg mice (n=8/genotype) and probed with the indicated antibodies. (FIG. 2B) Quantitative analysis of the blots shows that the levels of the NMDA receptor subunit NR2B phosphorylated at Tyr1472, PKA, and pERK were significantly decreased in the 3×Tg-AD mice compared to Non-Tg mice. Protein levels are expressed as fold changes over Non-Tg mice and represent means±SEM. * indicates p<0.05.

FIGS. 3A-D. CBP gene transfer rescues learning and memory deficit in 3×Tg-AD mice. CBP expressing lentiviruses were injected into the dorso-lateral ventricle of 3×Tg-AD (n=18) and Non-Tg (n=17). Additionally, 17 3×Tg-AD and 16 Non-Tg mice received sham injections. Six mice/group were sacrifice after 3 and 5 days of training. (FIG. 3A) All mice were evaluated in the spatial reference version of the MWM. The escape latency of the CBP-injected 3×Tg-AD mice was significantly lower than sham-injected 3×Tg-AD mice (p=0.008). (FIGS. 3B-D) Reference memory was significantly improved in CBP-injected 3×Tg-AD mice compared to sham-injected 3×Tg-AD mice in all probe-trial measurements conducted. Data are presented as means±SEM. * indicates p<0.05.

FIGS. 4A-C. CBP gene delivery restores pCREB levels after 5 days of training. (FIG. 4A) Representative Western blots of proteins extracted from the hippocampi of CBP- and sham-injected 3×Tg-AD and Non-Tg mice mice (n=6/group), and probed with the indicated antibodies. (FIGS. 4B-C) Quantitative analysis of the blots shows that CBP levels were significantly increased in both 3×Tg-AD and Non-Tg mice receiving the virus. In contrast, pCREB levels were significantly increased in the hippocampi of the CBP-injected compared to sham-injected 3×Tg-AD mice but not in the Non-Tg mice. Data are presented as fold changes over sham-injected Non-Tg mice and represent means±SEM. * indicates p<0.05.

FIGS. 5A-F. CBP gene delivery rescues BDNF levels. (FIG. 5A) Representative Western blots of proteins extracted from the hippocampi of CBP- and sham-injected 3×Tg-AD and Non-Tg mice and probed with an anti-BDNF antibody. (FIG. 5B) Quantitative analysis of the blots shows that in the hippocampi of the 3×Tg-AD mice, CBP gene delivery restored the levels of pro-BDNF and BDNF to Non-Tg levels. (FIG. 5C) Representative Western blots of proteins extracted from the hippocampi of 6-month-old sham- and CBP-injected 3×Tg-AD and Non-Tg mice (n=6/genotype) after 5 days of training, and probed with the indicated antibodies. (FIGS. 5D-F) Quantitative analysis of the blots shows that after 5 days of training, the levels of pNR2B, PKA and pERK were significantly increased in the CBP-injected 3×Tg-AD mice compared to sham-injected 3×Tg-AD mice. In contrast, no differences were found between sham- and CBP-injected Non-Tg mice. Data are presented as-fold changes over sham-injected Non-Tg mice and represent means±SEM. * indicates p<0.05.

FIGS. 6A-H. Aβ accumulation decreases CREB signaling in vivo. (FIG. 6A) Aβ levels measured by sandwich ELISA in the hippocampi of 6-month-old 3×Tg-AD mice (n=6) that received a single injection of 2 μg of 6E10 into the left hippocampi. The right un-injected hippocampi were used as internal controls. Mice were sacrificed 3 days after the antibody delivery. The levels of Aβ40 and Aβ42 were significantly lower in the left hippocampi receiving 6E10, compared to the right un-injected hippocampi. (FIG. 6B) Representative Western blots (probed with the indicated antibodies) of proteins extracted from the hippocampi of mice injected with 6E10 compared with the contralateral un-injected hippocampi. (FIG. 6C) Quantitative analyses of the blots showed that while CREB levels were similar between the ipsilateral hippocampi (receiving 6E10) and the contralateral un-injected hippocampi, reducing Aβ levels was sufficient to significantly increase pCREB levels (n=6). (FIG. 6D) Representative microphotograph of CA1-pyramidal neurons from the 3×Tg-AD and APP/tau mice stained with an Aβ42 specific antibody. (FIG. 6E) Representative Western blots of proteins extracted from the hippocampi of 6-month-old 3×Tg-AD and APP/tau mice (n=6/genotype) and probed with the indicated antibodies. (FIG. 6F) Quantitative analysis of the blots shows that the pCREB (but not total CREB) levels were significantly higher in the hippocampi of the APP/tau compared to 3×Tg-AD mice. (FIG. 6G) Representative Western blots of proteins extracted from the hippocampi of 2-month-old Non-Tg mice injected with CHO or 7PA2 condition medium, and with conditioned medium from 7PA2 cells depleted of Aβ by immunoprecipitation with 6E10 (n=6/group). (FIG. 6H) Quantitative analysis of the blots shows that pCREB (but not total CREB) levels were significantly reduced after injection of 7PA2 conditioned medium. Data represent means±SEM. * indicates p<0.05.

FIGS. 7A-F. Extent of viral diffusion. (FIG. 7A) Schematic representation of the plasmid used to generate the CBP-expressing lentivirus. The CBP gene was under the control of the neuronal specific EF1a promoter. An HA tag was added at the 3′-end of the CBP gene. (FIGS. 7B-C) Representative microphotographs depicting the dentate gyrus of sham-injected and CBP-injected mice, respectively. Sections were stained with an anti-HA antibody, showing a strong expression of the virus in this brain region. Notably, the * indicates the central ventricle. (FIG. 7D) Representative microphotographs of the CA1 pyramidal neurons (top two panels) and the cortex (bottom two panels) of sham- and CBP-injected 3×Tg-AD mice. Sections were stained with an anti-HA antibody and indicate that the virus injected CA1 pyramidal neurons. In contrast, the virus did not spread to cortical regions. (FIG. 7E) Representative microphotographs from sham- and CBP-injected 3×Tg-AD mice. The top two panels were stained with an anti-HA antibody (dark grey) and an anti-neurofilament antibody (light grey), and clearly show the expression of the HA tag in neurons. The bottom two panels were stained with an anti-HA antibody (dark grey) and an anti-GFAP antibody (light grey), and clearly show that the HA tag was not expressed in astrocytes. (FIG. 7F) Representative microphotographs depicting the contralateral and ipsilateral (relative to the injection site) dentate gyrus of CBP-injected Non-Tg mice. Sections were stained with an anti-HA antibody and show that the virus infected neurons on both hemi brains.

FIG. 8. Swimming speeds. Swimming speed was not significant across the four groups of mice as determined by 2-way ANOVA. Data are presented as means±SEM.

FIGS. 9A-E. CBP gene delivery restores pCREB levels. (FIG. 9A) Representative Western blots of proteins extracted from the hippocampi of CBP- and sham-injected 3×Tg-AD and Non-Tg mice (n=6/group) at baseline (7 days after the viral injection but without training in the water maze) and after 3 days of training. Blots were probed with the indicated antibodies. (FIGS. 9B-E) Quantitative analysis of the blots shows that at baseline (B) and after 3 days of training (D), CBP levels were significantly increased in the 3×Tg-AD and Non-Tg mice receiving the virus. In contrast, at both time-points, pCREB levels were significantly increased in the hippocampi of the CBP-injected compared to sham-injected 3×Tg-AD mice but not in the Non-Tg mice. Data are presented as fold changes over sham-injected Non-Tg mice and represent means±SEM. * indicates p<0.05.

FIGS. 10A-D. CBP gene delivery does not alter Aβ and tau pathology. (FIG. 10A) Representative microphotographs of CA1 pyramidal neurons from CBP-injected 3×Tg-AD mice. Sections were stained with the indicated antibodies and show that the HA tag is expressed in Aβ₄₂ and tau-bearing neurons. (FIG. 10B) Aβ levels were measured in the hippocampi of injected mice by sandwich ELISA and indicated that CBP gene delivery did not alter Aβ levels. (FIGS. 10C-D) Representative microphotographs of CA1-pyramidal neurons of CBP- and sham-injected 3×Tg-AD mice. The sections were immunostained with the reported antibodies and show that CBP-gene delivery did not change Aβ and tau immunoreactivity in the hippocampus. Data are presented as means±SEM.

FIGS. 11A-B. BDNF levels are lower in the hippocampi of the 3×Tg-AD mice. (FIG. 11A) Representative Western blots of proteins extracted from the hippocampi of 6-month-old 3×Tg-AD and Non-Tg mice (n=6/genotype) and probed with an anti-BDNF antibody. (FIG. 11B) Quantitative analysis of the blots shows that pro-BDNF and BDNF levels were significantly lower in the hippocampi of the 3×Tg-AD compared to Non-Tg mice. Data are presented as means±SEM. * indicates p<0.05.

FIGS. 12A-G. CBP gene delivery restored NDMA signaling. (FIG. 12A) Representative Western blots of proteins extracted from the hippocampi of CBP- and sham-injected 3×Tg-AD and Non-Tg mice (n=6/group) at baseline (7 days after the viral injection but without training in the water maze) and after 3 days of training. Blots were probed with the indicated antibodies. (FIGS. 12B-C) Quantitative analysis of the blots shows at baseline, the levels of pNR2B and PKA were significantly higher in the CBP-injected 3×tg-AD compared to sham-injected 3×Tg-AD mice. In contrast, no changes were detected when comparing the sham-injected Non-Tg mice to CBP-injected Non-Tg mice. (FIG. 12D) At baseline, ERK phosphorylation was not altered in the CBP-injected mice. (FIGS. 12E-G) The levels of pNR2B, PKA, and pERK were significantly increased in the CBP-injected 3×Tg-AD mice compared to sham-injected 3×Tg-AD mice after 3 days of training. In contrast, no differences were found between sham- and CBP-injected Non-Tg mice. Data are presented as fold changes over sham-injected Non-Tg mice and represent means SEM. * indicates p<0.05.

### DESCRIPTION OF ILLUSTRATIVE EMBODIMENTS

Neurodegenerative diseases are particularly devastating in that they progressively incapacitate their victims. Remarkably, though much progress has been made in recent years, there remain relatively few drugs that are useful in the treatment of neurodegenerative diseases, and almost none that are effective for a high percentage of patients. Thus, there is an urgent need for new and improved drugs and methods of therapy for these conditions, which includes neurodegenerative diseases such as Alzheimer's Disease, a condition that has devastating effects on cognitive function and overall mental health costing billions of dollars in healthcare for the elderly.

The inventor has now demonstrated that CREB-binding protein (CBP) is able to increase the neuronal level of the critical neurotrophin brain-derived neurotrophic factor (NBDF). The leads to improved memory and learing in an Alzheimer's Disease mouse model without affect tau morphology. These and other aspects of the invention are described in detail below.

### I. NEURODEGENERATIVE DISEASE

Neurodegeneration is the umbrella term for the progressive loss of structure or function of neurons, including death of neurons. Many neurodegenerative diseases including ALS, Parkinson's, Alzheimer's and Huntington's occur as a result of neurodegenerative processes. As research progresses, many similarities appear which relate these diseases to one another on a sub-cellular level. Discovering these similarities offers hope for therapeutic advances that could ameliorate many diseases simultaneously. There are many parallels between different neurodegenerative disorders including atypical protein assemblies as well as induced cell death. Neurodegeneration can be found in many different levels of neuronal circuitry ranging from molecular to systemic.

A. Alzheimer's Disease

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

B. Parkinson's Disease

Parkinson's disease (PD) is a degenerative disorder of the central nervous system. The motor symptoms of Parkinson's disease result from the death of dopamine-generating cells in the substantia nigra, a region of the midbrain; the cause of cell-death is unknown. Early in the course of the disease, the most obvious symptoms are movement-related, including shaking, rigidity, slowness of movement and difficulty with walking and gait. Later, cognitive and behavioural problems may arise, with dementia commonly occurring in the advanced stages of the disease. Other symptoms include sensory, sleep and emotional problems. PD is more common in the elderly with most cases occurring after the age of 50.

The main motor symptoms are collectively called parkinsonism, or a “parkinsonian syndrome.” Parkinson's disease is often defined as a parkinsonian syndrome that is idiopathic (having no known cause), although some atypical cases have a genetic origin. Many risk and protective factors have been investigated: the clearest evidence is for an increased risk of PD in people exposed to certain pesticides and a reduced risk in tobacco smokers. The pathology of the disease is characterized by the accumulation of a protein called alpha-synuclein into inclusions called Lewy bodies in neurons, and from insufficient formation and activity of dopamine produced in certain neurons within parts of the midbrain. Lewy bodies are the pathological hallmark of the idiopathic disorder and the distribution of the Lewy bodies throughout the Parkinsonian brain varies from one individual to another. The anatomical distribution of the Lewy body is often directly related to the expression and degree of the clinical symptoms of each individual. Diagnosis of typical cases is mainly based on symptoms, with tests such as neuroimaging being used for confirmation.

Modern treatments are effective at managing the early motor symptoms of the disease, mainly through the use of levodopa and dopamine agonists. As the disease progresses and dopamine neurons continue to be lost, a point eventually arrives at which these drugs become ineffective at treating the symptoms and at the same time produce a complication called dyskinesia, marked by involuntary writhing movements. Diet and some forms of rehabilitation have shown some effectiveness at alleviating symptoms. Surgery and deep brain stimulation have been used to reduce motor symptoms as a last resort in severe cases where drugs are ineffective. Research directions include a search of new animal models of the disease and investigations of the potential usefulness of gene therapy, stem cell transplants and neuroprotective agents. Medications to treat non-movement-related symptoms of PD, such as sleep disturbances and emotional problems, also exist.

The term parkinsonism is used for a motor syndrome whose main symptoms are tremor at rest, stiffness, slowing of movement and postural instability. Parkinsonian syndromes can be divided into four subtypes according to their origin: primary or idiopathic, secondary or acquired, hereditary parkinsonism, and parkinson plus syndromes or multiple system degeneration. Parkinson's disease is the most common form of parkinsonism and is usually defined as “primary” parkinsonism, meaning parkinsonism with no external identifiable cause. In recent years several genes that are directly related to some cases of Parkinson's disease have been discovered. As much as this can go against the definition of Parkinson's disease as an idiopathic illness, genetic parkinsonism disorders with a similar clinical course to PD are generally included under the Parkinson's disease label. The terms “familial Parkinson's disease” and “sporadic Parkinson's disease” can be used to differentiate genetic from truly idiopathic forms of the disease.

PD is usually classified as a movement disorder, although it also gives rise to several non-motor types of symptoms such as sensory deficits, cognitive difficulties or sleep problems. Parkinson plus diseases are primary parkinsonisms which present additional features. They include multiple system atrophy, progressive supranuclear palsy, corticobasal degeneration and dementia with Lewy bodies.

In terms of pathophysiology, PD is considered a synucleinopathy due to an abnormal accumulation of alpha-synuclein protein in the brain in the form of Lewy bodies, as opposed to other diseases such as Alzheimer's disease where the brain accumulates tau protein in the form of neurofibrillary tangles. Nevertheless, there is clinical and pathological overlap between tauopathies and synucleinopathies. The most typical symptom of Alzheimer's disease, dementia, occurs in advanced stages of PD, while it is common to find neurofibrillary tangles in brains affected by PD.

Dementia with Lewy bodies (DLB) is another synucleinopathy that has similarities with PD, and especially with the subset of PD cases with dementia. However the relationship between PD and DLB is complex and still has to be clarified. They may represent parts of a continuum or they may be separate diseases.

Four motor symptoms are considered cardinal in PD: tremor, rigidity, slowness of movement, and postural instability. Tremor is the most apparent and well-known symptom. It is the most common; though around 30% of individuals with PD do not have tremor at disease onset, most develop it as the disease progresses. It is usually a rest tremor: maximal when the limb is at rest and disappearing with voluntary movement and sleep. It affects to a greater extent the most distal part of the limb and at onset typically appears in only a single arm or leg, becoming bilateral later. Frequency of PD tremor is between 4 and 6 hertz (cycles per second). A feature of tremor is “pill-rolling,” a term used to describe the tendency of the index finger of the hand to get into contact with the thumb and perform together a circular movement. The term derives from the similarity between the movement in PD patients and the earlier pharmaceutical technique of manually making pills.

Bradykinesia (slowness of movement) is another characteristic feature of PD, and is associated with difficulties along the whole course of the movement process, from planning to initiation and finally execution of a movement. Performance of sequential and simultaneous movement is hindered. Bradykinesia is the most disabling symptom in the early stages of the disease. Initial manifestations are problems when performing daily tasks which require fine motor control such as writing, sewing or getting dressed. Clinical evaluation is based in similar tasks such as alternating movements between both hands or both feet. Bradykinesia is not equal for all movements or times. It is modified by the activity or emotional state of the subject, to the point that some patients are barely able to walk yet can still ride a bicycle. Generally patients have less difficulty when some sort of external cue is provided.

Rigidity is stiffness and resistance to limb movement caused by increased muscle tone, an excessive and continuous contraction of muscles. In parkinsonism the rigidity can be uniform (lead-pipe rigidity) or ratchety (cogwheel rigidity). The combination of tremor and increased tone is considered to be at the origin of cogwheel rigidity. Rigidity may be associated with joint pain; such pain being a frequent initial manifestation of the disease. In early stages of Parkinson's disease, rigidity is often asymmetrical and it tends to affect the neck and shoulder muscles prior to the muscles of the face and extremities. With the progression of the disease, rigidity typically affects the whole body and reduces the ability to move.

Postural instability is typical in the late stages of the disease, leading to impaired balance and frequent falls, and secondarily to bone fractures. Instability is often absent in the initial stages, especially in younger people. Up to 40% of the patients may experience falls and around 10% may have falls weekly, with number of falls being related to the severity of PD.

Other recognized motor signs and symptoms include gait and posture disturbances such as festination (rapid shuffling steps and a forward-flexed posture when walking), speech and swallowing disturbances including voice disorders, mask-like face expression or small handwriting, although the range of possible motor problems that can appear is large.

Parkinson's disease can cause neuropsychiatric disturbances which can range from mild to severe. This includes disorders of speech, cognition, mood, behaviour, and thought. Cognitive disturbances can occur in the initial stages of the disease and sometimes prior to diagnosis, and increase in prevalence with duration of the disease. The most common cognitive deficit in affected individuals is executive dysfunction, which can include problems with planning, cognitive flexibility, abstract thinking, rule acquisition, initiating appropriate actions and inhibiting inappropriate actions, and selecting relevant sensory information. Fluctuations in attention and slowed cognitive speed are among other cognitive difficulties. Memory is affected, specifically in recalling learned information. Nevertheless, improvement appears when recall is aided by cues. Visuospatial difficulties are also part of the disease, seen for example when the individual is asked to perform tests of facial recognition and perception of the orientation of drawn lines.

A person with PD has two to six times the risk of suffering dementia compared to the general population. The prevalence of dementia increases with duration of the disease. Dementia is associated with a reduced quality of life in people with PD and their caregivers, increased mortality, and a higher probability of needing nursing home care. Behavior and mood alterations are more common in PD without cognitive impairment than in the general population, and are usually present in PD with dementia. The most frequent mood difficulties are depression, apathy and anxiety. Impulse control behaviors such as medication overuse and craving, binge eating, hypersexuality, or pathological gambling can appear in PD and have been related to the medications used to manage the disease. Psychotic symptoms—hallucinations or delusions—occur in 4% of patients, and it is assumed that the main precipitant of psychotic phenomena in Parkinson's disease is dopaminergic excess secondary to treatment; it therefore becomes more common with increasing age and levodopa intake.

In addition to cognitive and motor symptoms, PD can impair other body functions. Sleep problems are a feature of the disease and can be worsened by medications. Symptoms can manifest in daytime drowsiness, disturbances in REM sleep, or insomnia. Alterations in the autonomic nervous system can lead to orthostatic hypotension (low blood pressure upon standing), oily skin and excessive sweating, urinary incontinence and altered sexual function. Constipation and gastric dysmotility can be severe enough to cause discomfort and even endanger health. PD is related to several eye and vision abnormalities such as decreased blink rate, dry eyes, deficient ocular pursuit (eye tracking) and saccadic movements (fast automatic movements of both eyes in the same direction), difficulties in directing gaze upward, and blurred or double vision. Changes in perception may include an impaired sense of smell, sensation of pain and paresthesia (skin tingling and numbness). All of these symptoms can occur years before diagnosis of the disease.

A physician will diagnose PD from the medical history and a neurological examination. There is no lab test that will clearly identify the disease, but brain scans are sometimes used to rule out disorders that could give rise to similar symptoms, Patients may be given levodopa and resulting relief of motor impairment tends to confirm diagnosis. The finding of Lewy bodies in the midbrain on autopsy is usually considered proof that the patient suffered from PD. The progress of the illness over time may reveal it is not PD, and some authorities recommend that the diagnosis be periodically reviewed.

Other causes that can secondarily produce a parkinsonian syndrome are Alzheimer's disease, multiple cerebral infarction and drug-induced parkinsonism. Parkinson plus syndromes such as progressive supranuclear palsy and multiple system atrophy must be ruled out. Anti-Parkinson's medications are typically less effective at controlling symptoms in Parkinson plus syndromes. Faster progression rates, early cognitive dysfunction or postural instability, minimal tremor or symmetry at onset may indicate a Parkinson plus disease rather than PD itself. Genetic forms are usually classified as PD, although the terms familial Parkinson's disease and familial parkinsonism are used for disease entities with an autosomal dominant or recessive pattern of inheritance.

Computed tomography (CT) and magnetic resonance imaging (MRI) brain scans of people with PD usually appear normal. These techniques are nevertheless useful to rule out other diseases that can be secondary causes of parkinsonism, such as basal ganglia tumors, vascular pathology and hydrocephalus. A specific technique of MRI, diffusion MRI, has been reported to be useful at discriminating between typical and atypical parkinsonism, although its exact diagnostic value is still under investigation. Dopaminergic function in the basal ganglia can be measured with different PET and SPECT radiotracers. Examples are ioflupane (123I) (trade name DaTSCAN) and iometopane (Dopascan) for SPECT or fludeoxyglucose (18F) for PET. A pattern of reduced dopaminergic activity in the basal ganglia can aid in diagnosing PD.

There is no cure for PD, but medications, surgery and multidisciplinary management can provide relief from the symptoms. The main families of drugs useful for treating motor symptoms are levodopa (usually combined with a dopa decarboxylase inhibitor or COMT inhibitor), dopamine agonists and MAO-B inhibitors. The stage of the disease determines which group is most useful. Two stages are usually distinguished: an initial stage in which the individual with PD has already developed some disability for which he needs pharmacological treatment, then a second stage in which an individual develops motor complications related to levodopa usage. Treatment in the initial stage aims for an optimal tradeoff between good symptom control and side-effects resulting from enhancement of dopaminergic function. The start of levodopa (or L-DOPA) treatment may be delayed by using other medications such as MAO-B inhibitors and dopamine agonists, in the hope of delaying the onset of dyskinesias. In the second stage the aim is to reduce symptoms while controlling fluctuations of the response to medication. Sudden withdrawals from medication or overuse have to be managed. When medications are not enough to control symptoms, surgery and deep brain stimulation can be of use. In the final stages of the disease, palliative care is provided to enhance quality of life.

Levodopa has been the most widely used treatment for over 30 years. L-DOPA is converted into dopamine in the dopaminergic neurons by dopa decarboxylase. Since motor symptoms are produced by a lack of dopamine in the substantia nigra, the administration of L-DOPA temporarily diminishes the motor symptoms. Only 5-10% of L-DOPA crosses the blood-brain barrier. The remainder is often metabolized to dopamine elsewhere, causing a variety of side effects including nausea, dyskinesias and joint stiffness. Carbidopa and benserazide are peripheral dopa decarboxylase inhibitors, which help to prevent the metabolism of L-DOPA before it reaches the dopaminergic neurons, therefore reducing side effects and increasing bioavailability. They are generally given as combination preparations with levodopa. Existing preparations are carbidopa/levodopa (co-careldopa) and benserazide/levodopa (co-beneldopa). Levodopa has been related to dopamine dysregulation syndrome, which is a compulsive overuse of the medication, and punding. There are controlled release versions of levodopa in the form intravenous and intestinal infusions that spread out the effect of the medication. These slow-release levodopa preparations have not shown an increased control of motor symptoms or motor complications when compared to immediate release preparations.

Tolcapone inhibits the COMT enzyme, which degrades dopamine, thereby prolonging the effects of levodopa. It has been used to complement levodopa; however, its usefulness is limited by possible side effects such as liver damage. A similarly effective drug, entacapone, has not been shown to cause significant alterations of liver function. Licensed preparations of entacapone contain entacapone alone or in combination with carbidopa and levodopa.

Levodopa preparations lead in the long term to the development of motor complications characterized by involuntary movements called dyskinesias and fluctuations in the response to medication. When this occurs a person with PD can change from phases with good response to medication and few symptoms (“on” state), to phases with no response to medication and significant motor symptoms (“off” state). For this reason, levodopa doses are kept as low as possible while maintaining functionality. Delaying the initiation of therapy with levodopa by using alternatives (dopamine agonists and MAO-B inhibitors) is common practice. A former strategy to reduce motor complications was to withdraw L-DOPA medication for some time. This is discouraged now, since it can bring dangerous side effects such as neuroleptic malignant syndrome. Most people with PD will eventually need levodopa and later develop motor side effects.

Several dopamine agonists that bind to dopaminergic post-synaptic receptors in the brain have similar effects to levodopa. These were initially used for individuals experiencing on-off fluctuations and dyskinesias as a complementary therapy to levodopa; they are now mainly used on their own as an initial therapy for motor symptoms with the aim of delaying motor complications. When used in late PD they are useful at reducing the off periods. Dopamine agonists include bromocriptine, pergolide, pramipexole, ropinirole, piribedil, cabergoline, apomorphine and lisuride.

Dopamine agonists produce significant, although usually mild, side effects including drowsiness, hallucinations, insomnia, nausea and constipation. Sometimes side effects appear even at a minimal clinically effective dose, leading the physician to search for a different drug. Compared with levodopa, dopamine agonists may delay motor complications of medication use but are less effective at controlling symptoms. Nevertheless, they are usually effective enough to manage symptoms in the initial years. They tend to be more expensive than levodopa. Dyskinesias due to dopamine agonists are rare in younger people who have PD, but along with other side effects, become more common with age at onset. Thus dopamine agonists are the preferred initial treatment for earlier onset, as opposed to levodopa in later onset. Agonists have been related to a impulse control disorders (such as compulsive sexual activity and eating, and pathological gambling and shopping) even more strongly than levodopa.

Apomorphine, a non-orally administered dopamine agonist, may be used to reduce off periods and dyskinesia in late PD. It is administered by intermittent injections or continuous subcutaneous infusions. Since secondary effects such as confusion and hallucinations are common, individuals receiving apomorphine treatment should be closely monitored. Two dopamine agonists that are administered through skin patches (lisuride and rotigotine) have been recently found to be useful for patients in initial stages and preliminary positive results has been published on the control of off states in patients in the advanced state.

MAO-B inhibitors (selegiline and rasagiline) increase the level of dopamine in the basal ganglia by blocking its metabolism. They inhibit monoamine oxidase-B (MAO-B) which breaks down dopamine secreted by the dopaminergic neurons. The reduction in MAO-B activity results in increased L-DOPA in the striatum. Like dopamine agonists, MAO-B inhibitors used as monotherapy improve motor symptoms and delay the need for levodopa in early disease, but produce more adverse effects and are less effective than levodopa. There are few studies of their effectiveness in the advanced stage, although results suggest that they are useful to reduce fluctuations between on and off periods. An initial study indicated that selegiline in combination with levodopa increased the risk of death, but this was later disproven.

Other drugs such as amantadine and anticholinergics may be useful as treatment of motor symptoms. However, the evidence supporting them lacks quality, so they are not first choice treatments. In addition to motor symptoms, PD is accompanied by a diverse range of symptoms. A number of drugs have been used to treat some of these problems. Examples are the use of clozapine for psychosis, cholinesterase inhibitors for dementia, and modafinil for daytime sleepiness. A 2010 meta-analysis found that non-steroidal anti-inflammatory drugs (apart from acetaminophen and aspirin), have been associated with at least a 15 percent (higher in long-term and regular users) reduction of incidence of the development of Parkinson's disease.

Placement of an electrode into the brain. The head is stabilised in a frame for stereotactic surgery. Treating motor symptoms with surgery was once a common practice, but since the discovery of levodopa, the number of operations declined. Studies in the past few decades have led to great improvements in surgical techniques, so that surgery is again being used in people with advanced PD for whom drug therapy is no longer sufficient. Surgery for PD can be divided in two main groups: lesional and deep brain stimulation (DBS). Target areas for DBS or lesions include the thalamus, the globus pallidus or the subthalamic nucleus. Deep brain stimulation (DBS) is the most commonly used surgical treatment. It involves the implantation of a medical device called a brain pacemaker, which sends electrical impulses to specific parts of the brain. DBS is recommended for people who have PD who suffer from motor fluctuations and tremor inadequately controlled by medication, or to those who are intolerant to medication, as long as they do not have severe neuropsychiatric problems. Other, less common, surgical therapies involve the formation of lesions in specific subcortical areas (a technique known as pallidotomy in the case of the lesion being produced in the globus pallidus).

There is some evidence that speech or mobility problems can improve with rehabilitation, although studies are scarce and of low quality. Regular physical exercise with or without physiotherapy can be beneficial to maintain and improve mobility, flexibility, strength, gait speed, and quality of life. However, when an exercise program is performed under the supervision of a physiotherapist, there are more improvements in motor symptoms, mental and emotional functions, daily living activities, and quality of life compared to a self-supervised exercise program at home. In terms of improving flexibility and range of motion for patients experiencing rigidity, generalized relaxation techniques such as gentle rocking have been found to decrease excessive muscle tension. Other effective techniques to promote relaxation include slow rotational movements of the extremities and trunk, rhythmic initiation, diaphragmatic breathing, and meditation techniques. As for gait and addressing the challenges associated with the disease such as hypokinesia (slowness of movement), shuffling and decreased arm swing; physiotherapists have a variety of strategies to improve functional mobility and safety. Areas of interest with respect to gait during rehabilitation programs focus on but are not limited to improving gait speed, base of support, stride length, trunk and arm swing movement. Strategies include utilizing assistive equipment (pole walking and treadmill walking), verbal cueing (manual, visual and auditory), exercises (marching and PNF patterns) and altering environments (surfaces, inputs, open vs. closed). Strengthening exercises have shown improvements in strength and motor function for patients with primary muscular weakness and weakness related to inactivity with mild to moderate Parkinson's disease. However, reports show a significant interaction between strength and the time the medications was taken. Therefore, it is recommended that patients should perform exercises 45 minutes to one hour after medications, when the patient is at their best. Also, due to the forward flexed posture, and respiratory dysfunctions in advanced PD, deep diaphragmatic breathing exercises are beneficial in improving chest wall mobility and vital capacity. Exercise may improve constipation.

Palliative care is often required in the final stages of the disease when all other treatment strategies have become ineffective. The aim of palliative care is to maximize the quality of life for the person with the disease and those surrounding him or her. Some central issues of palliative care are: care in the community while adequate care can be given there, reducing or withdrawing drug intake to reduce drug side effects, preventing pressure ulcers by management of pressure areas of inactive patients, and facilitating end-of-life decisions for the patient as well as involved friends and relatives.

C. ALS

Amyotrophic lateral sclerosis (ALS), sometimes called Lou Gehrig's Disease, affects as many as 20,000 Americans at any given time, with 5,000 new cases being diagnosed in the United States each year. ALS affects people of all races and ethnic backgrounds. Men are about 1.5 times more likely than women to be diagnosed with the disease. ALS strikes in the prime of life, with people most commonly diagnosed between the ages of 40 and 70. However, it is possible for individuals to be diagnosed at younger and older ages. About 90-95% of ALS cases occur at random, meaning that individuals do not have a family history of the disease and other family members are not at increased risk for contracting the disease. In about 5-10% of ALS cases there is a family history of the disease.

ALS is a progressive neurological disease that attacks neurons that control voluntary muscles. Motor neurons, which are lost in ALS, are specialized nerve cells located in the brain, brainstem, and spinal cord. These neurons serve as connections from the nervous system to the muscles in the body, and their function is necessary for normal muscle movement. ALS causes motor neurons in both the brain and spinal cord to degenerate, and thus lose the ability to initiate and send messages to the muscles in the body. When the muscles become unable to function, they gradually atrophy and twitch. ALS can begin with very subtle symptoms such as weakness in affected muscles. Where this weakness first appears differs for different people, but the weakness and atrophy spread to other parts of the body as the disease progresses.

Initial symptoms may affect only one leg or arm, causing awkwardness and stumbling when walking or running. Subjects also may suffer difficulty lifting objects or with tasks that require manual dexterity. Eventually, the individual will not be able to stand or walk or use hands and arms to perform activities of daily living. In later stages of the disease, when the muscles in the diaphragm and chest wall become too weak, patients require a ventilator to breathe. Most people with ALS die from respiratory failure, usually 3 to 5 years after being diagnosed; however, some people survive 10 or more years after diagnosis.

Perhaps the most tragic irony of ALS is that it does not impair a person's mind, as the disease affects only the motor neurons. Personality, intelligence, memory, and self-awareness are not affected, nor are the senses of sight, smell, touch, hearing, and taste. Yet at the same time, ALS causes dramatic defects in an individual's ability to speak loudly and clearly, and eventually, completely prevents speaking and vocalizing. Early speech-related symptoms include nasal speech quality, difficulty pronouncing words, and difficulty with conversation. As muscles for breathing weaken, it becomes difficult for patients to speak loud enough to be understood and, eventually, extensive muscle atrophy eliminates the ability to speak altogether. Patients also experience difficulty chewing and swallowing, which increase over time to the point that a feeding tube is required.

No test can provide a definite diagnosis of ALS, although the presence of upper and lower motor neuron signs in a single limb is strongly suggestive. Instead, the diagnosis of ALS is primarily based on the symptoms and signs the physician observes in the patient and a series of tests to rule out other diseases. Physicians obtain the patient's full medical history and usually conduct a neurologic examination at regular intervals to assess whether symptoms such as muscle weakness, atrophy of muscles, hyperreflexia, and spasticity are getting progressively worse.

MRI (axial FLAIR) demonstrates increased T2 signal within the posterior part of the internal capsule, consistent with the clinical diagnosis of ALS. Because symptoms of ALS can be similar to those of a wide variety of other, more treatable diseases or disorders, appropriate tests must be conducted to exclude the possibility of other conditions. One of these tests is electromyography (EMG), a special recording technique that detects electrical activity in muscles. Certain EMG findings can support the diagnosis of ALS. Another common test measures nerve conduction velocity (NCV). Specific abnormalities in the NCV results may suggest, for example, that the patient has a form of peripheral neuropathy (damage to peripheral nerves) or myopathy (muscle disease) rather than ALS. The physician may order magnetic resonance imaging (MRI), a noninvasive procedure that uses a magnetic field and radio waves to take detailed images of the brain and spinal cord. Although these MRI scans are often normal in patients with ALS, they can reveal evidence of other problems that may be causing the symptoms, such as a spinal cord tumor, multiple sclerosis, a herniated disk in the neck, syringomyelia, or cervical spondylosis.

Based on the patient's symptoms and findings from the examination and from these tests, the physician may order tests on blood and urine samples to eliminate the possibility of other diseases as well as routine laboratory tests. In some cases, for example, if a physician suspects that the patient may have a myopathy rather than ALS, a muscle biopsy may be performed. Because of the prognosis carried by this diagnosis and the variety of diseases or disorders that can resemble ALS in the early stages of the disease, patients should always obtain a second neurological opinion.

Riluzole (Rilutek®) as of 2011 is the only treatment that has been found to improve survival but only to a modest extent. It lengthens survival by several months, and may have a greater survival benefit for those with a bulbar onset. It also extends the time before a person needs ventilation support. Riluzole does not reverse the damage already done to motor neurons, and people taking it must be monitored for liver damage (occurring in ˜10% of people taking the drug).

Other treatments for ALS are designed to relieve symptoms and improve the quality of life for patients. This supportive care is best provided by multidisciplinary teams of health care professionals such as physicians; pharmacists; physical, occupational, and speech therapists; nutritionists; social workers; and home care and hospice nurses. Working with patients and caregivers, these teams can design an individualized plan of medical and physical therapy and provide special equipment aimed at keeping patients as mobile and comfortable as possible. Medical professionals can prescribe medications to help reduce fatigue, ease muscle cramps, control spasticity, and reduce excess saliva and phlegm. Drugs also are available to help patients with pain, depression, sleep disturbances, dysphagia, and constipation.

Physical therapists and occupational therapists playa large role in rehabilitation for individuals with ALS. Specifically, physical and occupational therapists can set goals and promote benefits for individuals with ALS by delaying loss of strength, maintaining endurance, limiting pain, preventing complications, and promoting functional independence. There is also a strong emphasis on the importance of patient and caregiver education that can be reinforced by physical therapists or occupational therapists. Research is controversial as to whether implementing a specific exercise program for these individuals may be beneficial; moreover, it is important for a physical therapist to address and understand the risks associated with implementing these types of programs for each and every person with ALS and the severity of their condition. The controversy lies in the fact that because ALS is characteristic of the degeneration of upper and lower motor neurons, that these neurons may react differently to specific exercise programs. Because spasticity is a common characteristic for individuals with ALS, physical therapists aim to reduce this by implementing range of motion activities with minimal resistance. In addition to range of motion activities, positioning techniques and splinting have also been shown to reduce spasticity; moreover, these techniques can also play an integral role in the reduction of pain for people with ALS. Overall, physical therapists have been proven to have positive effects on individuals with ALS by prescribing techniques and equipment to assist with conserving energy, emphasizing the importance of education, limiting pain, and help to maintain a level of function appropriate for each of their clients with ALS.

Occupational therapy and special equipment such as assistive technology can also enhance patients' independence and safety throughout the course of ALS. But physical therapists must be mindful when prescribing assistive devices, keeping in mind the patients and their attitudes. Devices should make the patient feel hopeful, not helpless. Gentle, low-impact aerobic exercise such as walking, swimming, and stationary bicycling can strengthen unaffected muscles, improve cardiovascular health, and help patients fight fatigue and depression. Range of motion and stretching exercises can help prevent painful spasticity and shortening (contracture) of muscles. Physical therapists can recommend exercises that provide these benefits without overworking muscles. They can suggest devices such as ramps, braces, walkers, and wheelchairs that help patients remain mobile. Examples of devices prescribed can include cervical collars. In ALS, there will be a progression of cervical extensor weakness. Weakness of the muscles will cause the patient's head to fall forward, leading to acute neck pain, potential for chronic cervical conditions to develop and tightness of anterior neck muscles. A forward head posture will interfere in patients ADLs, making them more dependent on caretakers. A cervical collar can help restore their independence and comfort. When there is mild to moderate weakness of the cervical extensor, the therapist may provide a soft foam collar. When more severe weakness is observed, a more rigid collar will be beneficial. Occupational therapists can provide or recommend equipment and adaptations to enable people to retain as much independence in activities of daily living as possible.

Eventually most people with ALS are not able to stand or walk, get in or out of bed on their own, use their hands and arms, or communicate. In later stages of the disease, individuals have difficulty breathing as the muscles of the respiratory system weaken. Although respiratory support can ease problems with breathing and prolong survival, it does not affect the progression of ALS. Most people with ALS die from respiratory failure, usually within three to five years from the onset of symptoms. The median survival time from onset to death ranges from 20 to 48 months, but 10 to 20% of ALS patients have a survival longer than 10 years.

ALS patients who have difficulty speaking may benefit from working with a speech-language pathologist. These health professionals can teach patients adaptive strategies such as techniques to help them speak louder and more clearly. As ALS progresses, speech-language pathologists can recommend the use of augmentative and alternative communication such as voice amplifiers, speech-generating devices (or voice output communication devices) and/or low tech communication techniques such as alphabet boards or yes/no signals. These methods and devices help patients communicate when they can no longer speak or produce vocal sounds. With the help of occupational therapists, speech-generating devices can be activated by switches or mouse emulation techniques controlled by small physical movements of, for example, the head, finger or eyes. In every case, the appropriate therapist should be mindful of the patients' preferences, attitudes, and likely progression over time.

Patients and caregivers can learn from speech-language pathologists and nutritionists how to plan and prepare numerous small meals throughout the day that provide enough calories, fiber, and fluid and how to avoid foods that are difficult to swallow. Patients may begin using suction devices to remove excess fluids or saliva and prevent choking. When patients can no longer get enough nourishment from eating, doctors may advise inserting a feeding tube into the stomach. The use of a feeding tube also reduces the risk of choking and pneumonia that can result from inhaling liquids into the lungs. The tube is not painful and does not prevent patients from eating food orally if they wish.

When the muscles that assist in breathing weaken, use of ventilatory assistance (intermittent positive pressure ventilation (IPPV), bilevel positive airway pressure (BIPAP), or biphasic cuirass ventilation (BCV)) may be used to aid breathing. Such devices artificially inflate the patient's lungs from various external sources that are applied directly to the face or body. When muscles are no longer able to maintain oxygen and carbon dioxide levels, these devices may be used full-time. BCV has the added advantage of being able to assist in clearing secretions by using high-frequency oscillations followed by several positive expiratory breaths. Patients may eventually consider forms of mechanical ventilation (respirators) in which a machine inflates and deflates the lungs. To be effective, this may require a tube that passes from the nose or mouth to the windpipe (trachea) and for long-term use, an operation such as a tracheostomy, in which a plastic breathing tube is inserted directly in the patient's windpipe through an opening in the neck.

Patients and their families should consider several factors when deciding whether and when to use one of these options. Ventilation devices differ in their effect on the patient's quality of life and in cost. Although ventilation support can ease problems with breathing and prolong survival, it does not affect the progression of ALS. Patients need to be fully informed about these considerations and the long-term effects of life without movement before they make decisions about ventilation support. Some patients under long-term tracheostomy intermittent positive pressure ventilation with deflated cuffs or cuffless tracheostomy tubes (leak ventilation) are able to speak, provided their bulbar muscles are strong enough. This technique preserves speech in some patients with long-term mechanical ventilation.

Social workers and home care and hospice nurses help patients, families, and caregivers with the medical, emotional, and financial challenges of coping with ALS, particularly during the final stages of the disease. Social workers provide support such as assistance in obtaining financial aid, arranging durable power of attorney, preparing a living will, and finding support groups for patients and caregivers. Home nurses are available not only to provide medical care but also to teach caregivers about tasks such as maintaining respirators, giving feedings, and moving patients to avoid painful skin problems and contractures. Home hospice nurses work in consultation with physicians to ensure proper medication, pain control, and other care affecting the quality of life of patients who wish to remain at home. The home hospice team can also counsel patients and caregivers about end-of-life issues.

D. Huntington's Disease

Huntington disease, also called Huntington's chorea, chorea major, or HD, is a genetic neurological disorder characterized by abnormal body movements called chorea and a lack of coordination; it also affects a number of mental abilities and some aspects of behavior. In 1993, the gene causing HD was found, making it one of the first inherited genetic disorders for which an accurate test could be performed. The accession number for Huntington is NM_{—}002111.

The gene causing the disorder is dominant and may, therefore, be inherited from a single parent. Global incidence varies, from 3 to 7 per 100,000 people of Western European descent, down to 1 per 1,000,000 of Asian and African descent. The onset of physical symptoms in HD occur in a large range around a mean of a person's late forties to early fifties. If symptoms become noticeable before a person is the age of twenty, then their condition is known as Juvenile HD.

A trinucleotide repeat expansion occurs in the Huntington gene, which produces mutant Huntington protein. The presence of this protein increases the rate of neuron cell death in select areas of the brain, affecting certain neurological functions. The loss of neurons isn't fatal, but complications caused by symptoms reduce life expectancy. There is currently no proven cure, so symptoms are managed with a range of medications and supportive services.

Symptoms increase in severity progressively, but are not often recognised until they reach certain stages. Physical symptoms are usually the first to cause problems and be noticed, but these are accompanied by cognitive and psychiatric ones which aren't often recognized. Almost everyone with HD eventually exhibits all physical symptoms, but cognitive symptoms vary, and so any psychopathological problems caused by these, also vary per individual. The symptoms of juvenile HD differ in that they generally progress faster and are more likely to exhibit rigidity and bradykinesia instead of chorea and often include seizures.

The most characteristic symptoms are jerky, random, and uncontrollable movements called chorea, although sometimes very slow movement and stiffness (bradykinesia, dystonia) can occur instead or in later stages. These abnormal movements are initially exhibited as general lack of coordination, an unsteady gait and slurring of speech. As the disease progresses, any function that requires muscle control is affected, this causes reduced physical stability, abnormal facial expression, impaired speech comprehensibility, and difficulties chewing and swallowing. Eating difficulties commonly cause weight loss. HD has been associated with sleep cycle disturbances, including insomnia and rapid eye movement sleep alterations.

Selective cognitive abilities are progressively impaired, including executive function (planning, cognitive flexibility, abstract thinking, rule acquisition, initiating appropriate actions and inhibiting inappropriate actions), psychomotor function (slowing of thought processes to control muscles), perceptual and spatial skills of self and surrounding environment, selection of correct methods of remembering information (but not actual memory itself), short-term memory, and ability to learn new skills, depending on the pathology of the individual.

Psychopathological symptoms vary more than cognitive and physical ones, and may include anxiety, depression, a reduced display of emotions (blunted affect) and decreased ability to recognize negative expressions like anger, disgust, fear or sadness in others, egocentrism, aggression, and compulsive behavior. The latter can cause, or worsen, hypersexuality and addictions such as alcoholism and gambling.

HD is autosomal dominant, needing only one affected allele from either parent to inherit the disease. Although this generally means there is a one in two chance of inheriting the disorder from an affected parent, the inheritance of HD is more complex due to potential dynamic mutations, where DNA replication does not produce an exact copy of itself. This can cause the number of repeats to change in successive generations. This can mean that a parent with a count close to the threshold, may pass on a gene with a count either side of the threshold. Repeat counts maternally inherited are usually similar, whereas paternally inherited ones tend to increase. This potential increase in repeats in successive generations is known as anticipation. In families where neither parent has HD, new mutations account for truly sporadic cases of the disease. The frequency of these de novo mutations is extremely low.

Homozygous individuals, who carry two mutated genes because both parents passed on one, are rare. While HD seemed to be the first disease for which homozygotes did not differ in clinical expression or course from typical heterozygotes, more recent analysis suggest that homozygosity affects the phenotype and the rate of disease progression though it does not alter the age of onset suggesting that the mechanisms underlying the onset and the progression are different.

Huntington protein is variable in its structure as there are many polymorphisms of the gene which can lead to variable numbers of glutamine residues present in the protein. In its wild-type (normal) form, it contains 6-35 glutamine residues; however, in individuals affected by HD, it contains between 36-155 glutamine residues. Huntington has a predicted mass of ˜350 kDa, however, this varies and is largely dependent on the number of glutamine residues in the protein. Normal huntingtin is generally accepted to be 3144 amino acids in size.

Two transcriptional pathways are more extensively implicated in HD—the CBP/p300 and Sp1 pathways—and these are transcription factors whose functions are vital for the expression of many genes. The postulated relationship between CBP and HD stems from studies showing that CBP is found in polyglutamine aggregates (see Kazantsev et al., 1999). Consequently, it was demonstrated that huntingtin and CBP interact via their polyglutamine stretches, that huntingtin with an expanded polyglutamine tract interferes with CBP-activated gene expression, and that overexpression of CBP rescued polyglutamine-induced toxicity in cultured cells (Nucifora et al., 2001; Steffan et al., 2001). Mutant huntingtin was also shown to interact with the acetyltransferase domain of CBP and inhibit the acetyltransferase activity of CBP, p300, and the p300/CBP-associated factor P/CAF (Steffan et al., 2001).

These observations prompted a hypothesis whereby the pathogenic process was linked to the state of histone acetylation; specifically, mutant huntingtin induced a state of decreased histone acetylation and thus altered gene expression. Support for this hypothesis was obtained in a Drosophila HD model expressing an N-terminal fragment of huntingtin with an expanded polyglutamine tract in the eye. Administration of inhibitors of histone deacetylase arrested the neurodegeneration and lethality (Steffan et al., 2001). Protective effects of HDAC inhibitors have been reported for other polyglutamine disorders, prompting the concept that at least some of the observed effects in polyglutamine disorders are due to alterations in histone acetylation (Hughes 2002). Studies published in 2002 revealed that the N-terminal fragment of huntingtin and intact huntingtin interact with Sp1 (Dunah et al., 2002; Li et al., 2002), a transcriptional activator that binds to upstream GC-rich elements in certain promoters. It is the glutamine-rich transactivation domain of Sp1 that selectively binds and directs core components of the general transcriptional complex such as TFIID, TBP and other TBP-associated factors to Sp1-dependent sites of transcription. In vitro transcription studies have gone on to show that in addition to targeting Sp1, mutant huntingtin targets TFIID and TFIIF, members of the core transcriptional complex (Zhai et al. 2005). Mutant huntingtin was shown to interact with the RAP30 subunit of TFIIF. Notably, overexpression of RAP30 alleviated both mutant huntingtin-induced toxicity and transcriptional repression of the dopamine D2 receptor gene. These results indicate that mutant huntingtin may interfere with multiple components of the transcription machinery.

There is no treatment to fully arrest the progression of the disease, but symptoms can be reduced or alleviated through the use of medication and care methods. Huntington mice models exposed to better husbandry techniques, especially better access to food and water, lived much longer than mice that were not well cared for.

Standard treatments to alleviate emotional symptoms include the use of antidepressants and sedatives, with antipsychotics (in low doses) for psychotic symptoms. Speech therapy helps by improving speech and swallowing methods; this therapy is more effective if started early on, as the ability to learn is reduced as the disease progresses. A two-year pilot study, of intensive speech, pyschiatric and physical therapy, applied to inpatient rehabilitation, showed motor decline was greatly reduced.

Nutrition is an important part of treatment; most third and fourth stage HD sufferers need two to three times the calories of the average person to maintain body weight. Healthier foods in pre-symptomatic and earlier stages may slow down the onset and progression of the disease. High calorie intake in pre-symptomatic and earlier stages has been shown to speed up the onset and reduce IQ level. Thickening agent can be added to drinks as swallowing becomes more difficult, as thicker fluids are easier and safer to swallow. The option of using a stomach PEG is available when eating becomes too hazardous or uncomfortable; this greatly reduces the chances of aspiration of food, and the subsequent increased risk of pneumonia, and increases the amount of nutrients and calories that can be ingested.

EPA, an Omega-3 fatty acid, may slow and possibly reverse the progression of the disease. As of April 2008, it is in FDA clinical trial as ethyl-EPA, (brand name Miraxion), for prescription use. Clinical trials utilise 2 grams per day of EPA. In the United States, it is available over the counter in lower concentrations in Omega-3 and fish oil supplements.

### II. CREB AND CREB-BINDING PROTEIN

A. CREB

CREB (cAMP response element-binding) is a cellular transcription factor. It binds to certain DNA sequences called cAMP response elements (CRE), thereby increasing or decreasing the transcription of the downstream genes. CREB was first described in 1987 as a cAMP-responsive transcription factor regulating the somatostatin gene. Genes whose transcription is regulated by CREB include: c-fos, the neurotrophin BDNF (Brain-derived neurotrophic factor), tyrosine hydroxylase, and many neuropeptides (such as somatostatin, enkephalin, VGF and corticotropin-releasing hormone).

CREB is closely related in structure and function to CREM (cAMP response element modulator) and ATF-1 (activating transcription factor-1) proteins. CREB proteins are expressed in many animals, including humans. CREB has a well-documented role in neuronal plasticity and long-term memory formation in the brain.

The cAMP response element is the response element for CREB. Since the effects of protein kinase A on the synthesis of proteins work by activating CREB, the cAMP response element is responsible for modulating the effects of protein kinase A that work by protein synthesis.

A typical (albeit somewhat simplified) sequence of events is as follows. A signal arrives at the cell surface, activates the corresponding receptor, which leads to the production of a second messenger such as cAMP or Ca²⁺, which in turn activates a protein kinase. This protein kinase translocates to the cell nucleus, where it activates a CREB protein. The activated CREB protein then binds to a CRE region, and is then bound to by a CBP (CREB-binding protein), which coactivates it, allowing it to switch certain genes on or off. The DNA binding of CREB is mediated via its basic leucine zipper domain (bZIP domain) as depicted in the picture.

CREB has many functions in many different organs, however most of its functions have been studied in relation to the brain. CREB proteins in neurons are thought to be involved in the formation of long-term memories; this has been shown in the marine snail Aplysia, the fruit fly Drosophila melanogaster, and in rats. CREB is necessary for the late stage of long-term potentiation. CREB also has an important role in the development of drug addiction. There are activator and repressor forms of CREB. Flies genetically engineered to overexpress the inactive form of CREB lose their ability to retain long-term memory. CREB is also important for the survival of neurons, as shown in genetically engineered mice, where CREB and CREM were deleted in the brain. If CREB is lost in the whole developing mouse embryo, the mice die immediately after birth, again highlighting the critical role of CREB in promoting survival.

Disturbance of CREB function in brain can contribute to the development and progression of Huntington's Disease. Abnormalities of a protein that interacts with the KID domain of CREB, the CREB-binding protein, (CBP) is associated with Rubinstein-Taybi syndrome. CREB is also thought to be involved in the growth of some types of cancer.

B. CREB-Binding Protein

CREB-binding protein, also known as CREBBP or CBP, is a protein that in humans is encoded by the CREBBP gene. The CREB protein carries out its function by activating transcription, where interaction with transcription factors is managed by one or more of p300 domains: the nuclear receptor interaction domain (RID), the CREB and MYB interaction domain (KIX), the cysteine/histidine regions (TAZ1/CH1 and TAZ2/CH3) and the interferon response binding domain (IBiD). The CREB protein domains, KIX, TAZ1 and TAZ2, each bind tightly to a sequence spanning both transactivation domains 9aaTADs of transcription factor p53. Mutations in this gene cause Rubinstein-Taybi syndrome (RTS). Chromosomal translocations involving this gene have been associated with acute myeloid leukemia.

This gene is ubiquitously expressed and is involved in the transcriptional coactivation of many different transcription factors. First isolated as a nuclear protein that binds to cAMP-response element-binding protein (CREB), this gene is now known to play critical roles in embryonic development, growth control, and homeostasis by coupling chromatin remodeling to transcription factor recognition. The protein encoded by this gene has intrinsic histone acetyltransferase activity and also acts as a scaffold to stabilize additional protein interactions with the transcription complex. This protein acetylates both histone and non-histone proteins. This protein shares regions of very high-sequence similarity with protein EP300 in its bromodomain, cysteine-histidine-rich regions, and histone acetyltransferase domain. Recent results suggest that novel CBP-mediated post-translational N-glycosylation activity alters the conformation of CBP-interacting proteins, leading to regulation of gene expression, cell growth and differentiation.

The mRNA sequence for CBP is shown below (SEQ ID NO:1):

| | |
|---|---|
| 1 | gctgttgctg aggctgagat ttggccgccg cctcccccac ccggcctgcg ccctccgcgg |
| 61 | cccggcccgc gctcctgcgc tcgctcctcg ctggctcgcc tgctcgcagc cgccggcccg |
| 121 | acccccgtcc gggccgcgtc gcgccgcccg cgctcagggc tgtttccgcg agcaggtgaa |
| 181 | gatggccgag aacttgctgg acggaccgcc caaccccaaa cgagccaaac tcagctcgcc |
| 241 | cggcttctcc gcgaatgaca acacagattt tggatcattg tttgacttgg aaaatgacct |
| 301 | tcctgatgag ctgatcccca atggagaatt aagcctttta aacagtggga accttgttcc |
| 361 | agatgctgcg tccaaacata aacaactgtc agagcttctt agaggaggca gcggctctag |
| 421 | catcaaccca gggataggca atgtgagtgc cagcagccct gtgcaacagg gccttggtgg |
| 481 | ccaggctcag gggcagccga acagtacaaa catggccagc ttaggtgcca tgggcaagag |
| 541 | ccctctgaac caaggagact catcaacacc caacctgccc aaacaggcag ccagcacctc |
| 601 | tgggcccact ccccctgcct cccaagcact gaatccacaa gcacaaaagc aagtagggct |
| 661 | ggtgaccagt agtcctgcca catcacagac tggacctggg atctgcatga atgctaactt |
| 721 | caaccagacc cacccaggcc ttctcaatag taactctggc catagcttaa tgaatcaggc |
| 781 | tcaacaaggg caagctcaag tcatgaatgg atctcttggg gctgctggaa gaggaagggg |
| 841 | agctggaatg ccctaccctg ctccagccat gcagggggcc acaagcagtg tgctggcgga |
| 901 | gaccttgaca caggtttccc cacaaatggc tggccatgct ggactaaata cagcacaggc |
| 961 | aggaggcatg accaagatgg gaatgactgg taccacaagt ccatttggac aaccctttag |
| 1021 | tcaaactgga gggcagcaga tgggagccac tggagtgaac ccccagttag ccagcaaaca |
| 1081 | gagcatggtc aatagtttac ctgcttttcc tacagatatc aagaatactt cagtcaccac |
| 1141 | tgtgccaaat atgtcccagt tgcaaacatc agtgggaatt gtacccacac aagcaattgc |
| 1201 | aacaggcccc acagcagacc ctgaaaaacg caaactgata cagcagcagc tggttctact |
| 1261 | gcttcatgcc cacaaatgtc agagacgaga gcaagcaaat ggagaggttc gagcctgttc |
| 1321 | tctcccacac tgtcgaacca tgaaaaacgt tttgaatcac atgacacatt gtcaggctgg |
| 1381 | gaaagcctgc caagttgccc attgtgcatc ttcacgacaa atcatctctc attggaagaa |
| 1441 | ctgcacacga catgactgtc ctgtttgcct ccctttgaaa aatgccagtg acaagcgaaa |
| 1501 | ccaacaaacc atcctgggat ctccagctag tggaattcaa aacacaattg gttctgttgg |
| 1561 | tgcagggcaa cagaatgcca cttccttaag taacccaaat cccatagacc ccagttccat |
| 1621 | gcagcgggcc tatgctgctc taggactccc ctacatgaac cagcctcaga cgcagctgca |
| 1681 | gcctcaggtt cctggccagc aaccagcaca gcctccagcc caccagcaga tgaggactct |
| 1741 | caatgcccta ggaaacaacc ccatgagtat cccagcagga ggaataacaa cagatcaaca |
| 1801 | gccaccaaac ttgatttcag aatcagctct tccaacttcc ttgggggcta ccaatccact |
| 1861 | gatgaatgat ggttcaaact ctggtaacat tggaagcctc agcacgatac ctacagcagc |
| 1921 | gcctccttcc agcactggtg ttcgaaaagg ctggcatgaa catgtgactc aggacctacg |
| 1981 | gagtcatcta gtccataaac tcgttcaagc catcttccca actccagacc ctgcagctct |
| 2041 | gaaagatcgc cgcatggaga acctggttgc ctatgctaag aaagtggagg gagacatgta |
| 2101 | tgagtctgct aatagcaggg atgaatacta tcatttatta gcagagaaaa tctataaaat |
| 2161 | acaaaaagaa ctagaagaaa agcggaggtc acgtttacat aagcaaggca tcctgggtaa |
| 2221 | ccagccagct ttaccagctt ctggggctca gccccctgtg attccaccag cccagtctgt |
| 2281 | aagacctcca aatgggcccc tgcctttgcc agtgaatcgc atgcaggttt ctcaagggat |
| 2341 | gaattcattt aacccaatgt ccctgggaaa cgtccagttg ccacaggcac ccatgggacc |
| 2401 | tcgtgcagcc tcccctatga accactctgt gcagatgaac agcatggcct cagttccggg |
| 2461 | tatggccatt tctccttcac ggatgcctca gcctccaaat atgatgggca ctcatgccaa |
| 2521 | caacattatg gcccaggcac ctactcagaa ccagtttctg ccacagaacc agtttccatc |
| 2581 | atccagtggg gcaatgagtg tgaacagtgt gggcatgggg caaccagcag cccaggcagg |
| 2641 | tgtttcacag ggtcaggtac ctggagctgc tctccctaac cctctgaaca tgctggcacc |
| 2701 | ccaggccagc cagctgcctt gcccaccagt gacacagtca ccattgcacc cgactccacc |
| 2761 | tcctgcttcc acagctgctg gcatgccctc tctccaacat ccaacggcac caggaatgac |
| 2821 | ccctcctcag ccagcagctc ccactcagcc atctactcct gtgtcatctg ggcagactcc |
| 2881 | taccccaact cctggctcag tgcccagcgc tgcccaaaca cagagtaccc ctacagtcca |
| 2941 | ggcagcagca caggctcagg tgactccaca gcctcagacc ccagtgcagc caccatctgt |
| 3001 | ggctactcct cagtcatcac agcagcaacc aacgcctgtg catactcagc ctcctggcac |
| 3061 | accgctttct caggcagcag ccagcattga taatagagtc cctactccct cctctgtgac |
| 3121 | cagtgctgaa accagttccc agcagccagg acccgatgtg cccatgctgg aaatgaagac |
| 3181 | agaggtgcag acagatgatg ctgagcctga acctactgaa tccaaggggg aacctcggtc |
| 3241 | tgagatgatg gaagaggatt tacaaggttc ttcccaagta aaagaagaga cagatacgac |
| 3301 | agagcagaag tcagagccaa tggaagtaga agaaaagaaa cctgaagtaa aagtggaagc |
| 3361 | taaagaggaa gaagagaaca gttcgaacga cacagcctca caatcaacat ctccttccca |
| 3421 | gccacgcaaa aaaatcttta aacccgagga gctacgccag gcacttatgc caactctaga |
| 3481 | agcactctat cgacaggacc cagagtcttt gccttttcgt cagcctgtag atcctcagct |
| 3541 | cctaggaatc ccagattatt ttgatatagt gaagaatcct atggaccttt ctaccatcaa |
| 3601 | acgaaagctg gacacagggc aatatcaaga accctggcag tatgtggatg atgtctggct |
| 3661 | tatgttcaac aatgcgtggc tatataatcg taaaacgtcc cgtgtatata aattttgcag |
| 3721 | taaacttgca gaggtctttg aacaagaaat tgaccctgtc atgcagtctc ttggatattg |
| 3781 | ctgtggacga aagtatgagt tctccccaca gactttgtgc tgttacggaa agcagctgtg |
| 3841 | tacaattcct cgtgatgcag cctactacag ctatcagaat aggtatcatt tctgtgagaa |
| 3901 | gtgtttcaca gagatccagg gcgagaatgt gaccctgggt gacgaccctt cccaacctca |
| 3961 | gacgacaatt tccaaggatc aatttgaaaa gaagaaaaat gataccttag atcctgaacc |
| 4021 | ttttgttgac tgcaaagagt gtggccggaa gatgcatcag atttgtgttc tacactatga |
| 4081 | catcatttgg ccttcaggtt ttgtgtgtga caactgtttg aagaaaactg gcagacctcg |
| 4141 | gaaagaaaac aaattcagtg ctaagaggct gcagaccaca cgattgggaa accacttaga |
| 4201 | agacagagtg aataagtttt tgcggcgcca gaatcaccct gaagctgggg aggtttttgt |
| 4261 | cagagtggtg gccagctcag acaagactgt ggaggtcaag ccgggaatga agtcaaggtt |
| 4321 | tgtggattct ggagagatgt cggaatcttt cccatatcgt accaaagcac tctttgcttt |
| 4381 | tgaggagatc gatggagtcg atgtgtgctt ttttgggatg catgtgcaag aatacggctc |
| 4441 | tgattgcccc ccaccaaata caaggcgtgt atacatatct tatctggaca gtattcattt |
| 4501 | cttccggccc cgctgcctcc ggacagctgt ttaccatgag atcctcatcg gatatctcga |
| 4561 | gtatgtgaag aaattggggt atgtgacagg acatatttgg gcctgtcccc caagtgaagg |
| 4621 | agatgactat atctttcatt gccacccccc tgaccagaaa atccccaaac caaaacgact |
| 4681 | acaggagtgg tacaagaaga tgctggacaa ggcgtttgca gagaggatca ttaacgacta |
| 4741 | taaggacatc ttcaaacaag cgaacgaaga caggctcacg agtgccaagg agttgcccta |
| 4801 | ttttgaagga gatttctggc ctaatgtgtt ggaagaaagc attaaggaac tagaacaaga |
| 4861 | agaagaagaa aggaaaaaag aagagagtac tgcagcgagt gagactcctg agggcagtca |
| 4921 | gggtgacagc aaaaatgcga agaaaaagaa caacaagaag accaacaaaa acaaaagcag |
| 4981 | cattagccgc gccaacaaga agaagcccag catgcccaat gtttccaacg acctgtcgca |
| 5041 | gaagctgtat gccaccatgg agaagcacaa ggaggtattc tttgtgattc atctgcatgc |
| 5101 | tgggcctgtt atcagcactc agccccccat cgtggaccct gatcctctgc ttagctgtga |
| 5161 | cctcatggat gggcgagatg ccttcctcac cctggccaga gacaagcact gggaattctc |
| 5221 | ttccttacgc cgctccaaat ggtccactct gtgcatgctg gtggagctgc acacacaggg |
| 5281 | ccaggaccgc tttgtttata cctgcaatga gtgcaaacac catgtggaaa cacgctggca |
| 5341 | ctgcactgtg tgtgaggact atgacctttg tatcaattgc tacaacacaa agagccacac |
| 5401 | ccataagatg gtgaagtggg ggctaggcct agatgatgag ggcagcagtc agggtgagcc |
| 5461 | acagtccaag agcccccagg aatcccggcg tctcagcatc cagcgctgca tccagtccct |
| 5521 | ggtgcatgcc tgccagtgtc gcaatgccaa ctgctcactg ccgtcttgcc agaagatgaa |
| 5581 | gcgagtcgtg cagcacacca agggctgcaa gcgcaagact aatggaggat gcccagtgtg |
| 5641 | caagcagctc attgctcttt gctgctacca cgccaaacac tgccaagaaa ataaatgccc |
| 5701 | tgtgcccttc tgcctcaaca tcaaacataa gctccgccag cagcagatcc agcatcgcct |
| 5761 | gcagcaggct cagctcatgc gccggcgaat ggcaaccatg aacacccgca atgtgcctca |
| 5821 | gcagagtttg ccttctccta cctcagcacc acccgggact cctacacagc agcccagcac |
| 5881 | accccaaaca ccacagcccc cagcccagcc tcagccttca cctgttaaca tgtcaccagc |
| 5941 | tggcttccct aatgtagccc ggactcagcc cccaacaata gtgtctgctg ggaagcctac |
| 6001 | caaccaggtg ccagctcccc caccccctgc ccagccccca cctgcagcag tagaagcagc |
| 6061 | ccggcaaatt gaacgtgagg cccagcagca gcagcaccta taccgagcaa acatcaacaa |
| 6121 | tggcatgccc ccaggacgtg caggtatggg gaccccagga agccaaatga ctcctgtggg |
| 6181 | cctgaatgtg ccccgtccca accaagtcag tgggcctgtc atgtctagta tgccacctgg |
| 6241 | gcagtggcag caggcaccca tccctcagca gcagccgatg ccaggcatgc ccaggcctgt |
| 6301 | aatgtccatg caggcccagg cagcagtggc tgggccacgg atgcccaatg tgcagccacc |
| 6361 | aaggagcatc tcgccaagtg ccctgcaaga cctgctacgg accctaaagt cacccagctc |
| 6421 | tcctcagcag cagcagcagg tgctgaacat ccttaaatca aacccacagc taatggcagc |
| 6481 | tttcatcaaa cagcgcacag ccaagtatgt ggccaatcag cctggcatgc agccccagcc |
| 6541 | cggacttcaa tcccagcctg gtatgcagcc ccagcctggc atgcaccagc agcctagttt |
| 6601 | gcaaaacctg aacgcaatgc aagctggtgt gccacggcct ggtgtgcctc caccacaacc |
| 6661 | agcaatggga ggcctgaatc cccagggaca agctctgaac atcatgaacc caggacacaa |
| 6721 | ccccaacatg acaaacatga atccacagta ccgagaaatg gtgaggagac agctgctaca |
| 6781 | gcaccagcag cagcagcagc aacagcagca gcagcagcag caacaacaaa atagtgccag |
| 6841 | cttggccggg ggcatggcgg gacacagcca gttccagcag ccacaaggac ctggaggtta |
| 6901 | tgccccagcc atgcagcagc aacgcatgca acagcacctc cccatccagg gcagctccat |
| 6961 | gggccagatg gctgctccaa tgggacaact tggccagatg gggcagcctg ggctaggggc |
| 7021 | agacagcacc cctaatatcc agcaggccct gcagcaacgg attctgcagc agcagcagat |
| 7081 | gaagcaacaa attgggtcac caggccagcc gaaccccatg agcccccagc agcacatgct |
| 7141 | ctcaggacag ccacaggcct cacatctccc tggccagcag atcgccacat cccttagtaa |
| 7201 | ccaggtgcga tctccagccc ctgtgcagtc tccacggccc caatcccaac ctccacattc |
| 7261 | cagcccgtca ccacggatac aaccccagcc ttcaccacac catgtttcac cccagactgg |
| 7321 | ttcccctcac cctggactcg cagtcaccat ggccagctcc atggatcagg gacacctggg |
| 7381 | gaaccctgaa cagagtgcaa tgctccccca gctgaatacc cccaacagga gcgcactgtc |
| 7441 | cagtgaactg tccctggttg gtgataccac gggagacaca ctagaaaagt ttgtggaggg |
| 7501 | tttgtag |

C. BDNF

Brain-derived neurotrophic factor, also known as BDNF, is a protein that, in humans, is encoded by the BDNF gene. BDNF is a member of the “neurotrophin” family of growth factors, which are related to the canonical “Nerve Growth Factor,” NGF. Neurotrophic factors are found in the brain and the periphery. BDNF acts on certain neurons of the central nervous system and the peripheral nervous system, helping to support the survival of existing neurons, and encourage the growth and differentiation of new neurons and synapses. In the brain, it is active in the hippocampus, cortex, and basal forebrain—areas vital to learning, memory, and higher thinking. BDNF itself is important for long-term memory. BDNF was the second neurotrophic factor to be characterized after nerve growth factor (NGF).

Although the vast majority of neurons in the mammalian brain are formed prenatally, parts of the adult brain retain the ability to grow new neurons from neural stem cells in a process known as neurogenesis. Neurotrophins are chemicals that help to stimulate and control neurogenesis, BDNF being one of the most active. Mice born without the ability to make BDNF suffer developmental defects in the brain and sensory nervous system, and usually die soon after birth, suggesting that BDNF plays an important role in normal neural development.

Despite its name, BDNF is actually found in a range of tissue and cell types, not just in the brain. It is also expressed in the retina, the central nervous system, motor neurons, the kidneys, and the prostate. BDNF is present in high concentration in hippocampus and cerebral cortex. BDNF is also found in human saliva.

BDNF binds at least two receptors on the surface of cells that are capable of responding to this growth factor, TrkB and the LNGFR (for low-affinity nerve growth factor receptor, also known as p75). It may also modulate the activity of various neurotransmitter receptors, including the Alpha-7 nicotinic receptor.

TrkB is a receptor tyrosine kinase (meaning it mediates its actions by causing the addition of phosphate molecules on certain tyrosines in the cell, activating cellular signaling). There are other related Trk receptors, TrkA and TrkC. Also, there are other neurotrophic factors structurally related to BDNF: NGF (for Nerve Growth Factor), NT-3 (for Neurotrophin-3) and NT-4 (for Neurotrophin-4). While TrkB mediates the effects of BDNF and NT-4, TrkA binds and is activated by NGF, and TrkC binds and is activated by NT-3. NT-3 binds to TrkA and TrkB as well, but with less affinity.

The other BDNF receptor, the p75, plays a somewhat less clear role. Some researchers have shown that the p75NTR binds and serves as a “sink” for neurotrophins. Cells that express both the p75NTR and the Trk receptors might, therefore, have a greater activity, since they have a higher “microconcentration” of the neurotrophin. It has also been shown, however, that the p75NTR may signal a cell to die via apoptosis; so, therefore, cells expressing the p75NTR in the absence of Trk receptors may die rather than live in the presence of a neurotrophin.

BDNF is made in the endoplasmic reticulum and secreted from dense-core vesicles. It binds carboxypeptidase E (CPE), and the disruption of this binding has been proposed to cause the loss of sorting of BDNF into dense-core vesicles. The phenotype for BDNF knockout mice can be severe, including postnatal lethality. Other traits include sensory neuron losses that affect coordination, balance, hearing, taste, and breathing. Knockout mice also exhibit cerebellar abnormalities and an increase in the number of sympathetic neurons. Exercise has been shown to increase the secretion of BDNF at the mRNA and protein levels in the rodent hippocampus, suggesting the potential increase of this neurotrophin after exercise in humans.

The BDNF protein is coded by the gene that is also called BDNF. In humans this gene is located on chromosome 11. Val66Met (rs6265) is a single nucleotide polymorphism in the gene where adenine and guanine alleles vary, resulting in a variation between valine and methionine at codon 66.

As of 2008, Val66Met is probably the most investigated SNP of the BDNF gene, but, besides this variant, other SNPs in the gene are C270T, rs7103411, rs2030324, rs2203877, rs2049045 and rs7124442. The polymorphism Thr2Ile may be linked to congenital central hypoventilation syndrome. In 2009, variants close to the BDNF gene were found to be associated with obesity in two very large genome wide-association studies of body mass index (BMI). Various studies have shown possible links between BDNF and conditions, such as depression, schizophrenia, obsessive-compulsive disorder, Alzheimer's disease, Huntington's disease, Rett syndrome, and dementia, as well as anorexia nervosa and bulimia nervosa. Increased levels of BDNF can induce a change to an opiate-dependent-like reward state when expressed in the ventral tegmental area in rats.

Exposure to stress and the stress hormone corticosterone has been shown to decrease the expression of BDNF in rats, and, if exposure is persistent, this leads to an eventual atrophy of the hippocampus. Atrophy of the hippocampus and other limbic structures has been shown to take place in humans suffering from chronic depression. In addition, rats bred to be heterozygous for BDNF, therefore reducing its expression, have been observed to exhibit similar hippocampal atrophy. This suggests that an etiological link between the development of depression and BDNF exists. Supporting this, the excitatory neurotransmitter glutamate, voluntary exercise, caloric restriction, intellectual stimulation, curcumin and various treatments for depression (such as antidepressants and electroconvulsive therapy and sleep deprivation) increase expression of BDNF in the brain. In the case of some treatments such as drugs and electroconvulsive therapy this has been shown to protect or reverse this atrophy.

Epilepsy has also been linked with polymorphisms in BDNF. Given BDNF's vital role in the development of the landscape of the brain, there is quite a lot of room for influence on the development of neuropathologies from BDNF. Levels of both BDNF mRNA and BDNF protein are known to be up-regulated in epilepsy. BDNF modulates excitatory and inhibitory synaptic transmission by inhibiting GABAA-receptor-mediated post-synaptic currents. This provides a potential mechanism for the observed up-regulation.

Post-mortem analysis has shown lowered levels of BDNF in the brain tissues of people with Alzheimer's disease, although the nature of the connection remains unclear. Studies suggest that neurotrophic factors have a protective role against amyloid beta toxicity.

### III. THERAPEUTIC REGIMENS

A. Therapeutic Agents

In accordance with the present invention, CBP will be delivered to cells using a vector capable of expressing CBP. The following is a general discussion of gene delivery for use in accordance with this goal.

1. Expression Constructs

Within certain embodiments expression vectors are employed to express a CBP polypeptide product. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the CBR polypeptide.

The term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

2. Viral Delivery

The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Of particular interest in the present invention are neurotrophic viruses such as herpesviruses, lentiviruses, adenoviruses and adeno-associated viruses. U.S. Pat. Nos. 6,344,445, 5,626,850, 5,223,424, 6,319,703 discuss herpesviruses vectors; U.S. Pat. Nos. 6,924,144, 6,521,457, 6,428,953, 6,277,633, 6,165,782 and 5,994,136 discuss lentivirus vectors; U.S. Pat. No. 7,094,398 discusses adenovirus vectors. Also contemplated are promoters known to be active in neuronal cells, such as EF1a, alpha-synuclein, beta-actin.

3. Non-Viral Delivery

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

Recent advances in lipid formulations have improved the efficiency of gene transfer in vivo (Smyth-Templeton et al., 1997, WO 98/07408). A lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

In further embodiments, the liposome is further defined as a nanoparticle. A “nanoparticle” is defined herein to refer to a submicron particle. The submicron particle can be of any size. For example, the nanoparticle may have a diameter of from about 0.1, 1, 10, 100, 300, 500, 700, 1000 nanometers or greater. The nanoparticles that are administered to a subject may be of more than one size.

Any method known to those of ordinary skill in the art can be used to produce nanoparticles. In some embodiments, the nanoparticles are extruded during the production process. Exemplary information pertaining to the production of nanoparticles can be found in U.S. Patent App. Pub. No. 20050143336, U.S. Patent App. Pub. No. 20030223938, U.S. Patent App. Pub. No. 20030147966, and U.S. Provisional Application Ser. No. 60/661,680, each of which is herein specifically incorporated by reference into this section.

B. Combinations Therapies

In another embodiment, the CBP therapy of the present invention may be used in combination with other agents to improve or enhance the therapeutic effect of either. This process may involve administering both agents to the patient at the same time, either as a single composition or pharmacological formulation that includes both agents, or by administering two distinct compositions or formulations, wherein one composition provides the CRBCBP therapy and the other provides the second agent(s).

The CBP therapy also may precede or follow the other agent treatment by intervals ranging from minutes to weeks. In embodiments where the other agent and CBP therapy are administered separately, one may prefer that a significant period of time did not expire between the time of each delivery, such that the other agent and CBP therapy would still be able to exert an advantageously combined effect. In such instances, it is contemplated that one may administer both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other embodiments, it may be desirable to alternate the compositions so that the subject is not tolerized.

Various additional combinations may be employed, CBP therapy is “A” and the other agent is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

It is expected that the treatment cycles would be repeated as necessary.

Various drugs for the treatment of AD are currently available as well as under study and regulatory consideration. The drugs generally fit into the broad categories of cholinesterase inhibitors, muscarinic agonists, anti-oxidants or anti-inflammatories. Galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol and neuromidal are just some of the drugs proposed as therapeutic agents for AD. Drugs for treating HD, ALS and PD are mentioned above and their discussion is incorporated here for the purpose of combination therapies.

### IV. PHARMACEUTICAL FORMULATIONS AND ROUTES OF ADMINISTRATION

Pharmaceutical compositions of the present invention comprise an effective amount of CBP or a CBP-expressing vector and/or additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains the CBP or a CBP-expressing vector or additional active ingredient will be known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered intracranially, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularlly, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 18^{th} Ed. Mack Printing Company, 1990, incorporated herein by reference).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the compounds of the present invention are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

The injectable composition may be administered into the brain of a subject through various techniques, including stereotactic surgery, stereotactic injection, stereotactic infusion, or convection-enhanced infusion. Various types of stereotactic frame systems may be utilized to facilitate administration of the composition. Various techniques can be used to facilitate localization and guidance of composition administration, including radiography, computed tomography, and magnetic resonance imaging.

### V. IDENTIFYING SUBJECTS HAVING AD AND MONITORING AD THERAPIES

In various aspects of the invention, it will be desirable to identify subjects that have AD. The general approaches for diagnosis is set out below. It also may be desirable to identify those individuals having increased risk for AD. At present, there are no truly prognostic tests. However, any of the following diagnostic procedures may be applied to individuals with few or no overt symptoms of AD and, in this way, provide early treatment that may prevent related neuropathologic damage and/or progression of the disease to a more clinically significant stage.

In various aspects of the invention, it will be desirable to identify subjects that have AD. The general approaches for diagnosis of these diseases are set out below. It also may be desirable to identify those individuals having increased risk for AD. At present, there are no truly prognostic tests. However, any of the following diagnostic procedures may be applied to individuals with few or no overt symptoms of AD and, in this way, provide early treatment that may prevent related neuropathologic damage and/or progression of the disease to a more clinically significant stage.

The diagnosis of both early (mild) cognitive impairment and AD are based primarily on clinical judgment. However, a variety of neuropsychological tests aid the clinician in reaching a diagnosis. Early detection of only memory deficits may be helpful in suggesting early signs of AD, since other dementias may present with memory deficits and other signs. Cognitive performance tests that assess early global cognitive dysfunction are useful, as well as measures of working memory, episodic memory, semantic memory, perceptual speed and visuospatial ability. These tests can be administered clinically, alone or in combination. Examples of cognitive tests according to cognitive domain are shown as examples, and include “Digits Backward” and “Symbol Digit” (Attention), “Word List Recall” and “Word List Recognition” (Memory), “Boston Naming” and “Category Fluency” (Language), “MMSE 1-10” (Orientation), and “Line Orientation” (Visuospatial). Thus, neuropsychological tests and education-adjusted ratings are assessed in combination with data on effort, education, occupation, and motor and sensory deficits. Since there are no consensus criteria to clinically diagnose mild cognitive impairment, various combinations of the above plus the clinical examination by an experienced neuropsychologist or neurologist are key to proper diagnosis. As the disease becomes more manifest (i.e., becomes a dementia rather than mild cognitive impairment), the clinician may use the criteria for dementia and AD set out by the joint working group of the National Institute of Neurologic and Communicative Disorders and Stroke/AD and Related Disorders Association (NINCDS/ADRDA). On occasion, a clinician may request a head computed tomography (CT) or a head magnetic resonance imaging (MRI) to assess degree of lobar atrophy, although this is not a requirement for the clinical diagnosis.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Virus and Surgical Procedures.

Mice were anesthetized with avertin (1.3% tribromoethanol, 0.8% amyl alcohol, given 0.6 ml/25 g body weight) and placed in a stereotactic apparatus. Mice received either sham injections of PBS or 5 μl of the CBP expressing lentivirus (˜1.2×10⁶ transducing particles/ml, purchased from BioGenova), which was injected into the dorso-lateral ventricle through a 33-gauge injector attached to a 5 μl Hamilton syringe. The coordinates, with respect to bregma, were −0.34 mm posterior, −2.2 mm lateral, and −1 ventral to the skull. Injections occurred over 5 min, after which the cannula was left in place for an additional 5 min to allow for diffusion. Animals were kept on a warming pad until they had fully recovered from anesthesia.

Mice and Behavioral Tests.

The derivation and characterization of the 3×Tg-AD and the APP/tau mice has been described elsewhere (Cowansage et al., 2010; Hock et al., 2000). The 3×Tg-AD and the Non-Tg mice used in these studies are on a mixed C57B16/129 background. Mice were group housed and kept on a 12 hour light:12 hour dark schedule. All animal procedures were in accordance with the National Institute of Health Guide for the Care and Use of Laboratory Animals, and all appropriate measures were taken to minimize pain and discomfort in experimental animals.

Spatial learning and memory were assessed using the Morris water maze tests, which were conducted in a circular tank of 1.5 meters in diameter located in a room with extra maze cues. The platform (14 cm in diameter) location was kept constant for each mouse during training and was 1.5 cm beneath the surface of the water, which was maintained at 25° C. throughout the duration of the testing. During 5 days of training the mice received 4 trials a day alternated among 4 pseudorandom starting points. If a mouse failed to find the platform within 60 seconds, it was guided to the platform by the researcher and kept there for 20 seconds. The inter-trial interval was 25 sec, during which each mouse was returned to its home cage. Probe trials were conducted 24 hours after the last training trial. During the probe trials, the platform was removed and mice were free to swim in the tank for 60 seconds. The training and probe trials were recorded by a video camera mounted on the ceiling, and data were analyzed using the EthoVisioXT tracking system.

Protein Extraction, Western-Blot.

Mice were sacrificed by CO₂ asphyxiation and their brains extracted and cut in-half sagitally. For immunohistochemical analysis, one-half was dropped-fixed in 4% paraformaldehyde in PBS for 48 hours and then transferred in 0.02% sodium azide in PBS until slicing. The other half was frozen in dry ice for biochemical analysis. Frozen brains were homogenized in a solution of tissue protein extraction reagent (T-PER, Pierce, Rockford, Ill.) containing 0.7 mg/ml Pepstatin A supplemented with a complete Mini protease inhibitor tablet (Roche, Switzerland) and phosphatase inhibitors (Invitrogen, Carlsbad, Calif.). The homogenized mixes were briefly sonicated to sheer the DNA and centrifuged at 4° C. for 1 hour at 100,000 g. The supernatant was stored as the soluble fraction. The pellet was re-homogenized in 70% formic acid and centrifuged as above. The supernatant was stored as the insoluble fraction.

For Western blot analyses, proteins from the soluble fraction were resolved by 10% Bis-Tris SDS/PAGE (Invitrogen, Carlsbad, Calif.) under reducing conditions and transferred to a nitrocellulose membrane. The membrane was incubated in a 5% solution of non-fat milk for 1 hour at 20° C. After overnight incubation at 4° C. with primary antibody, the blots were washed in Tween 20-TBS (T-TBS) (0.02% Tween 20, 100 mM Tris pH 7.5; 150 nM NaCl) for 20 minutes and incubated at 20° C. with secondary antibody. The blots were washed in T-TBS for 20 minutes and incubated for 5 minutes with Super Signal (Pierce), washed again and exposed.

ELISA.

Aβ1-40 and Aβ 1-42 levels were measured using a sensitive sandwich ELISA system. Proteins from the soluble fraction (see above) were loaded directly onto ELISA plates. MaxiSorp immunoplates (Nalge Nunc international, Rochester, N.Y.) were coated with monoclonal antibody 20.1, a specific antibody against Aβ₁₋₁₆ (from Dr. David Cribbs, University of California, Irvine) in coating buffer (0.1M NaCO₃ pH 9.6), and blocked with 3% BSA. Synthetic Aβ standards (Bachem, King of Prussia, Pa.) were defibrillated by dissolving in HFIP at 1 mg/ml and the HFIP evaporated with a stream of N₂. The defibrillated Aβ was then dissolved in DMSO at 1 mg/ml. Standards of both Aβ1-40 and Aβ1-42 were made in antigen capture buffer (ACB; 20 mM NAH₂PO₄, 2 mM EDTA, 0.4M NaCl, 0.5 g CHAPS, 1% BSA, pH 7.0), and loaded onto ELISA plates in duplicate. Samples were then loaded in duplicate and incubated overnight at 4° C. Plates were washed and probed with either HRP-conjugated anti-Aβ 35-40 (MM32-13.1.1, for Aβ₄₀) or anti-Aβ 35-42 (MM40-21.3.4, for β₄₂) overnight at 4° C. 3,3′,5,5′-tetramethylbenzidine was used as the chromagen, and the reaction was stopped with the addition of 30% O-phosphoric acid, and read at 450 nm on a plate reader (Labsystems, Sunnyvale, Calif.).

Immunohistochemistry.

For immunohistochemical analysis, 30 μm-thick sections were obtained using a vibratome slicing system (Leica Microsistems, Wetzlar, Germany) and sections were stored at 4° C. in 0.02% sodium azide in PBS. To quench the endogenous peroxidase activity, free-floating sections were incubated for 30 min in H₂O₂. The appropriate primary antibody was applied, and sections were incubated overnight at 4° C. Subsequently, sections were washed and incubated in the appropriate secondary antibody for 1 hour at 20° C. After a final wash of 20 min, sections were developed with diaminobenzidine (DAB) substrate using the avidin-biotin horseradish peroxidase system (Vector Laboratories).

Conditioned Medium.

7PA2 cells were grown in DMEM with 10% fetal bovine serum at 37° C. with humidified environment (5% CO₂) in 75 cm² cell culture flasks until 90% confluent. At this time, the medium was replaced with 7 ml of serum-free DMEM for 18 hours. Conditioned medium was then centrifuged to remove debris and frozen at −80° C. The medium was concentrated using Amicon Ultra-15 centrifuge filters (Millipore, Billerica, Mass.) with a 3 k molecular weight cut-off.

Statistical Analyses.

Learning data were analyzed using two-way analysis of variance (ANOVA). Specifically, the R statistical language was used with the NLME package to perform the mixed-model repeated measures ANOVA. The following model formula was used for the fixed effects: escape latency˜group+day+group:day where ‘group’ is a categorical covariate with four levels: Non-Tg-sham, 3×Tg-AD-sham, Non-Tg-CBP, and 3×Tg-AD-CBP (the first level was used as a baseline against which the other three levels were compared in the post-hoc test conducted with a Bonferroni correction); ‘day’ is a numeric covariate; and ‘group:day’ is the interaction term representing the effect of membership in a given experimental group on the slope of the escape latency over the five sessions. The random effect was animal ID, a categorical covariate distinguishing individual animals on which the repeated measures were performed. Probe trials were analyzed by one-way ANOVA following by post hoc Bonferroni test to determine individual differences in groups. Student T-test was used when suitable.

### Example 2

### Results

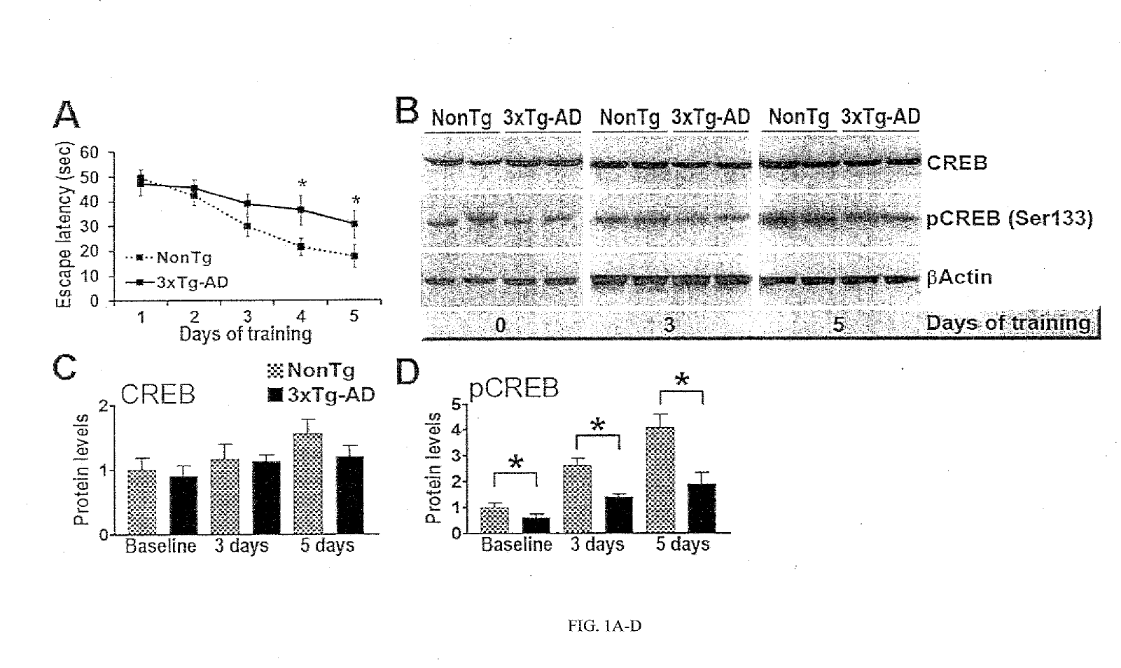

CREB phosphorylation and activity increases upon neuronal stimulation; such an activity-dependent increase is thought to facilitate the transcription of proteins required for learning and memory (Lee and Silva, 2009). Studies from various laboratories report that the expression of CREB is reduced in mouse models of AD, yet it is unclear whether and how CREB responds to neuronal stimulation in the presence of Aβ. To address these questions, the inventor used the 3×Tg-AD mice, a widely used animal model that develops Aβ and tau accumulation associated with cognitive dysfunction (Oddo et al., 2008; Oddo et al., 2003). The inventor trained 6-month-old 3×Tg-AD and Non-Tg mice (n=16/genotype) for either 3 or 5 days in the spatial version of the Morris water maze (MWM). At 6 months of age, the 3×Tg-AD mice show early synaptic and learning and memory dysfunction associated with the buildup of soluble Aβ levels (Oddo et al., 2008). Eight 3×Tg-AD and 8 Non-Tg mice were killed within 30 min of their last learning trial on day 3, and their hippocampi were removed and frozen in dry ice. The remaining mice received two additional days of training and their hippocampi were also removed and frozen within 30 min of their last training trial. A two-way ANOVA indicated a significant genotype (p<0.0001) and day effect (p<0.0001). The interaction genotype:day was also significant (p=0.0049; FIG. 1A). A Bonferroni post hoc analysis indicated that although no statistically significant differences between the two genotypes were detected after 3 days of training, the inventor found a strong trend supporting better performance by the Non-Tg mice (p=0.07) compared to the 3×Tg-AD mice (FIG. 1A). After 4 and 5 days of training, however, the average escape latency between Non-Tg and 3×Tg-AD mice was significantly different (p<0.01 and p<0.05, respectively; FIG. 1A). At the end of the learning trials, the levels of CREB in these mice were compared to mice that were sacrificed directly from their housing cages (n=8/genotype). The inventor found that total CREB levels were not statistically different between 3×Tg-AD and Non-Tg mice at any of the time-points analyzed (FIGS. 1B-C). In contrast, the steady-state levels of CREB phosphorylated at Ser133 (referred to as pCREB) in the hippocampi of the 3×Tg-AD mice were decreased by ˜40% compared to age- and gender-matched Non-Tg mice at baseline (i.e., prior to any MWM training; FIGS. 1B, 1D). Furthermore, the inventor found that pCREB levels in the Non-Tg mice increased as a function of training (FIG. 1B, D). In contrast, while pCREB levels in the 3×Tg-AD tended to increase with learning, these changes were not statistically significant (FIGS. 1B, 1D). When the data were analyzed as percentage change within each genotype, the inventor found a 423.8±134.2% increase in the Non-Tg mice over 5 days of training and a smaller but not significantly different increase in pCREB levels the 3×Tg-AD mice (305.6±142.9%). Nevertheless, at any of the time-point analyzed, pCREB levels were always significantly lower in the 3×Tg-AD mice. Furthermore, while at base line, pCREB levels were ˜40% lower in the hippocampi of the 3×Tg-AD mice compared to Non-Tg mice (FIG. 1D), after 3 and 5 days of MWM training, the pCREB levels were two-fold higher in the hippocampi of the Non-Tg mice compared to 3×Tg-AD mice (FIG. 1D). The hippocampi of 6-month-old 3×Tg-AD mice are characterized by the accumulation of Aβ and early tau mislocalization (Oddo et al., 2008), suggesting that the alterations in CREB phosphorylation may be due to Aβ accumulation and/or tau mislocalization.

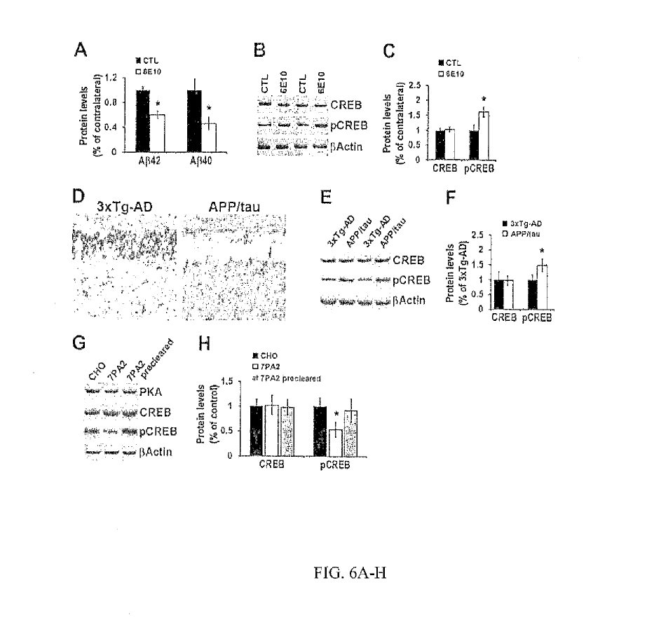

The inventor next sought to determine whether the changes in CREB phosphorylation in the hippocampi of the 3×Tg-AD mice are mediated by Aβ accumulation. Toward this end, the inventor first used an immunological approach to clear Aβ deposits from the brain of the 3×Tg-AD mice. Specifically, the inventor has shown that intrahippocampal injection of anti-Aβ antibodies is sufficient to clear Aβ deposits from the brains of the 3×Tg-AD mice (Oddo et al., 2004). The 6E10 antibody (2 μg) was stereotaxically injected into the left hippocampi of 6-month-old 3×Tg-AD mice; the contralateral un-injected hippocampi were used as internal controls. Three days post-injection, the hippocampi were removed and analyzed. Using sandwich ELISA, the inventor found that both soluble Aβ40 and Aβ42 levels were significantly reduced in the ipsilateral hippocampi (receiving 6E10) compared to the contralateral un-injected hippocampi (FIG. 6A). Notably, the reduction in Aβ levels led to a significant increase in pCREB, without affecting total CREB levels (FIGS. 6B-C). To further understand the relationship between Aβ and the CREB phosphorylation, the inventor used a genetic approach to prevent Aβ accumulation in the brains of the 3×Tg-AD mice. Toward this end, the inventor previously showed that replacing the mutant PS1 gene with its wild-type counterpart in the 3×Tg-AD mice is sufficient to prevent Aβ accumulation (Oddo et al., 2008). Indeed, in these mice (known as APP/tau), there was no Aβ immunoreactivity in the brain (FIG. 6D), despite that the APP and tau transgenes levels were unchanged (Oddo et al., 2008). Western blot analysis indicated that in APP/tau mice, which lack Aβ pathology, the levels of pCREB were significantly higher compared to age- and gender-matched 3×Tg-AD mice (FIGS. 6E-F).

To directly test for a causal relation between Aβ and CREB deficits, the inventor used Chinese hamster ovary (CHO) cells stably transfected with a cDNA encoding APP₇₅₁ containing the Val717Phe familial AD mutation (Koo and Squazzo, 1994). These cells, known as 7PA2, are widely used as they secrete high levels of Aβ oligomers, which cause cognitive dysfunction (Cleary et al., 2005). To determine whether Aβ oligomers affect CREB phosphorylation function in vivo, 7PA2 condition medium (CM) was concentrated by 50-fold using Amicon Ultra centrifugal filters and stereotaxically injected into the left hippocampi of 2-month-old Non-Tg mice (n=6); the right contralateral hippocampi were used as an internal control. Additional control groups were represented by mice injected with CM prepared from control CHO cells (n=6), and by mice injected with 7PA2 CM immunodepleted with 6E10 (n=6). Three days post-injection, mice were sacrificed and their hippocampi used for biochemical assessments. The inventor found that the levels of pCREB (but not total CREB), were significantly decreased in the ipsilateral-injected hippocampi compared to the contralateral un-injected hippocampi (FIGS. 6G-H). Notably, the injection of CM from CHO cells or 7PA2 CM that was depleted of Aβ by immunoprecipitation with 6E 10 had no effect on CREB phosphorylation (FIGS. 6G-H). Taken together, these results show that in 3×Tg-AD mice, the alterations in CREB phosphorylation are due to Aβ accumulation.

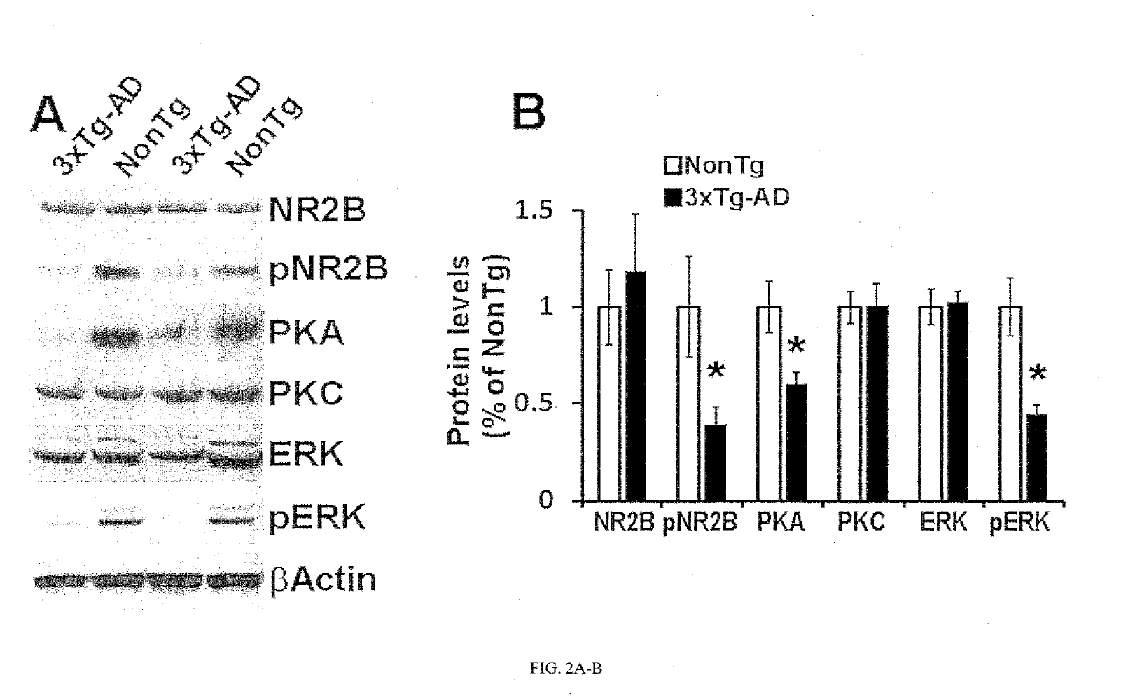

Evidence shows that Aβ accumulation reduces glutamatergic transmission (Palop and Mucke, 2010). Notably, during memory formation, CREB is phosphorylated at Ser133 via activation of NMDA receptors (Lee and Silva, 2009), suggesting that alteration in NMDA signaling may account for the decrease in pCREB levels in the 3×Tg-AD mice. To test this hypothesis, the inventor measured the steady-state levels of NMDA receptor subunit NR2B by Western blot and found no significant differences between 3×Tg-AD and Non-Tg mice (FIGS. 2A-B). However, the inventor found that the levels of NR2B phosphorylated at Tyr1472 were significantly reduced in the hippocampi of the 3×Tg-AD mice compared to Non-Tg mice (FIGS. 2A-B). This is highly noteworthy because reduced phosphorylation of NR2B at Thr1472 correlates with receptor endocytosis, which leads to reduced NMDA receptor signaling (Snyder et al., 2005).

Protein kinase A (PKA), protein kinase C (PKC), and the extracellular signal-regulated kinase (ERK) are three proteins downstream of NMDA signaling known to phosphorylate and activate CREB (Lee and Silva, 2009). The inventor thus measured the steady-state levels of these proteins in the hippocampi of 6-month-old mice and found that PKC levels were not different between 3×Tg-AD and Non-Tg mice (FIGS. 2A-B). In contrast, the inventor found that PKA levels were significantly decreased in the hippocampi of the 3×Tg-AD mice compared to Non-Tg mice (FIGS. 2A-B). Additionally, the inventor found that the levels of ERK phosphorylated at Thr202/204 (pERK), where significantly reduced in the 3×Tg-AD mice (FIGS. 2A-B). Taken together, these data show that two signaling pathways, downstream of NMDA, are deregulated in the hippocampi of 6-month-old 3×Tg-AD mice, and hence may account for the reduced CREB phosphorylation.

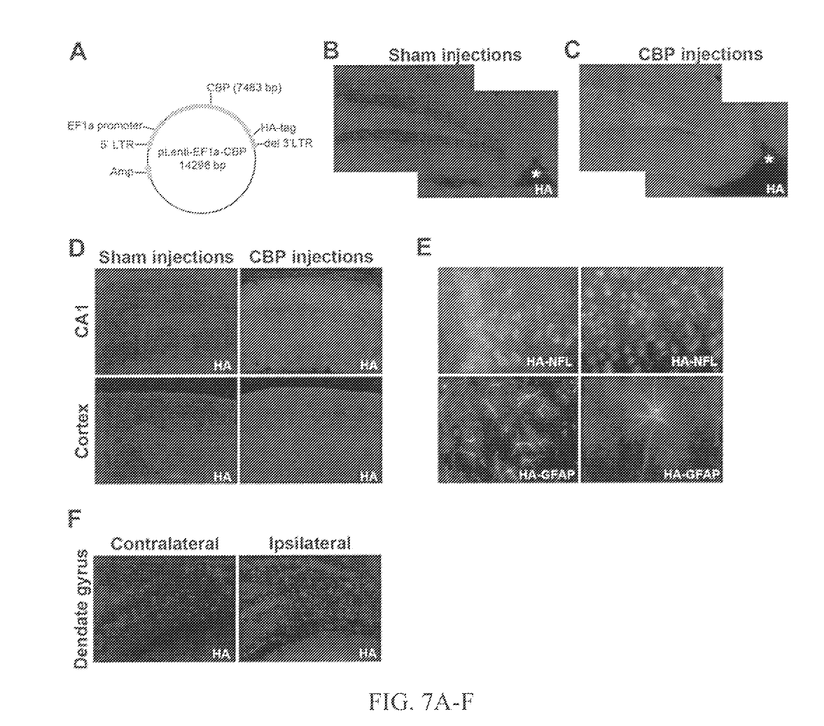

Considering the role of CREB in learning and memory, these data suggest that one way by which Aβ accumulation may induce cognitive deficits is by altering CREB phosphorylation and activity. CREB-binding protein (CBP) plays a critical role in stimulus-induced activation of CREB (Vo and Goodman, 2001); the inventor thus sought to facilitate CREB function in the brains of the 3×Tg-AD mice by overexpressing CREB-binding protein (CBP) using a lentiviral delivery system. The inventor generated a lentivirus expressing HA-tagged CBP under the control of the neuronal-specific promoter, EF1a (FIG. 7A). The inventor stereotaxically injected the CBP lentivirus into the left dorsal lateral ventricle of 6-month-old 3×Tg-AD mice (n=18) and Non-Tg mice (n=17). Additionally, 3×Tg-AD mice (n=17) and Non-Tg mice (n=16) received sham injections. The injection site was chosen based on previous reports showing that a viral vector injected into the lateral ventricle diffuses through the hippocampus (Wang et al., 2010). To determine the extent of the viral diffusion, the inventor stained sections from the sham- and CBP-injected mice with an anti-HA antibody and found only background staining in the hippocampi of the sham-injected mice (FIG. 7B). In contrast, the inventor found high HA immunoreactivity in the dentate gyrus and CA1 regions of the hippocampus in the CBP-injected mice (FIGS. 7C-D). Very low HA immunoreactivity was found in the cortex (FIG. 7D). Double-staining of sections from the CBP-injected mice confirmed that the virus was expressed mainly in neurons and not astrocytes (FIG. 7E). Notably, the inventor found that the virus also was expressed in the contralateral hippocampus (FIG. 7F).

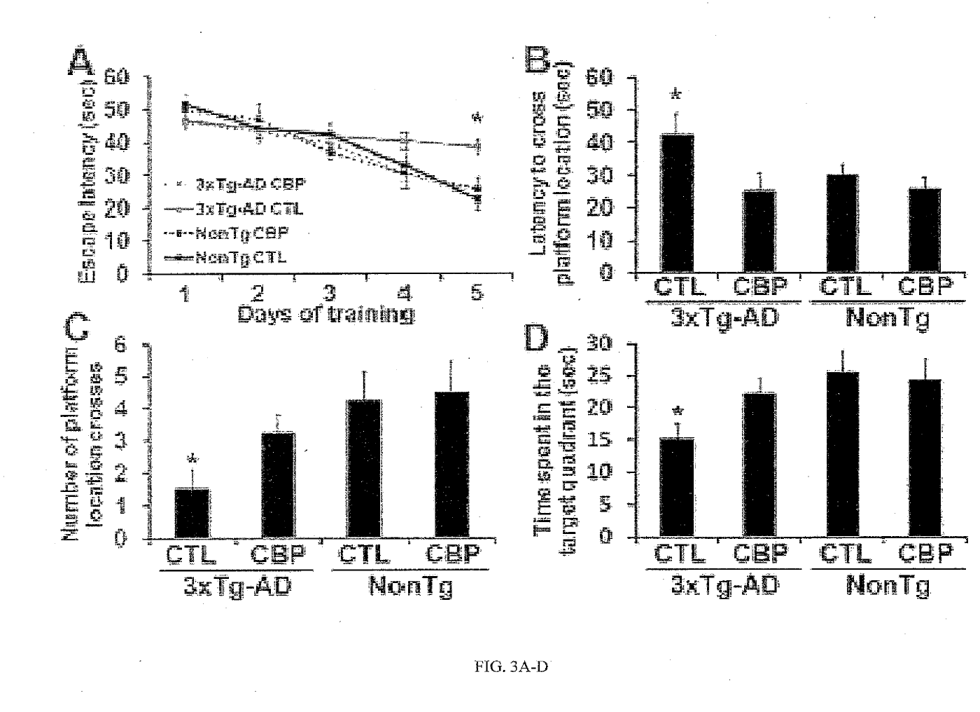

Seven days after the CBP delivery, all mice were tested using the MWM. Some of the mice were killed at 3 and 5 days after training (n=4/group/time-point) and their hippocampi extracted and processed for biochemical measurements (see below). The remaining mice were used to conduct the probe trials. To analyze the learning data, a mixed-model repeated measures ANOVA was used with treatment and genotype as categorically fixed effects, days as a numeric covariate, and animals as the random effect; escape latency was the dependent variable. The inventor found a significant effect for days (F=86.2541, p<0.0001), indicating that the slope of escape latency across the acquisition sessions was significantly different from zero (FIG. 3A). Furthermore, the group:days interaction was significant (F=4.5334; p=0.004091), indicating that one or more of the groups were different from each other. To find the group(s) most responsible for the differences, the inventor performed a post-hoc test with a Bonferroni correction and compared each of the individual interaction levels (i.e., slopes) to the sham-injected Non-Tg mice. The inventor found that only the sham-injected 3×Tg-AD mice had a significantly shallower slope than the sham-injected Non-Tg mice used as baseline group, indicating slower improvement in escape latency across the acquisition sessions (p=0.0049; FIG. 3A). These data indicate that the sham-injected 3×Tg-AD mice perform significantly worse that the sham-injected Non-Tg mice but the overexpression of CBP rescued the learning deficits of the 3×Tg-AD mice (as evidenced by the slope of the CBP-injected 3×Tg-AD mice not being significantly different from the sham-injected Non-Tg mice used as baseline); indeed, the CBP-injected 3×Tg-AD mice perform as well as the sham-injected Non-Tg mice (p=0.389; FIG. 3A). The slope of the escape latency of the CBP-injected Non-Tg mice was not significantly different from the sham-injected mice, indicating that CBP injections did not have any effects of the escape latency of the Non-Tg mice (FIG. 3A).

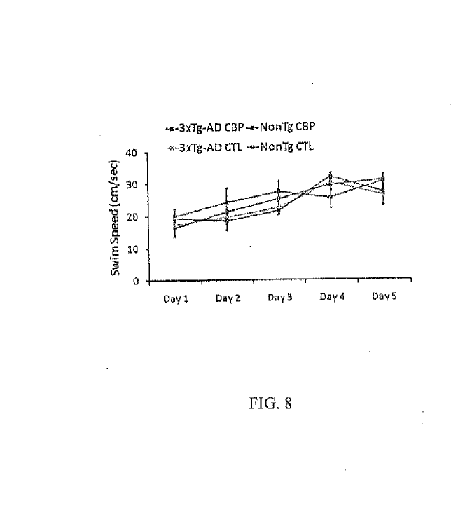

To measure spatial memory, probe trials were conducted 24 hours after the last training trial. One-way analysis of variance indicated a significant changes in the time that the mice took to cross the platform location (p=0.04, FIG. 3B), the number of platform location crosses during the 60-sec trials (p=0.03; FIG. 3C), and in the time the mice spent in the target quadrant (p=0.04; FIG. 3D). A post-hoc test with a Bonferroni correction showed that the CBP-injected 3×Tg-AD mice performed significantly worse than the sham-injected CBP mice in all three measurements (p<0.05). As for the learning trials, CBP gene delivery did not alter spatial memory in the Non-Tg mice (FIGS. 3B-D). Notably, the swimming speed was similar amongst all the mice used (FIG. 8). Taken together, these data clearly indicate that CBP gene delivery rescued learning and memory deficits in 6-month-old 3×Tg-AD mice.

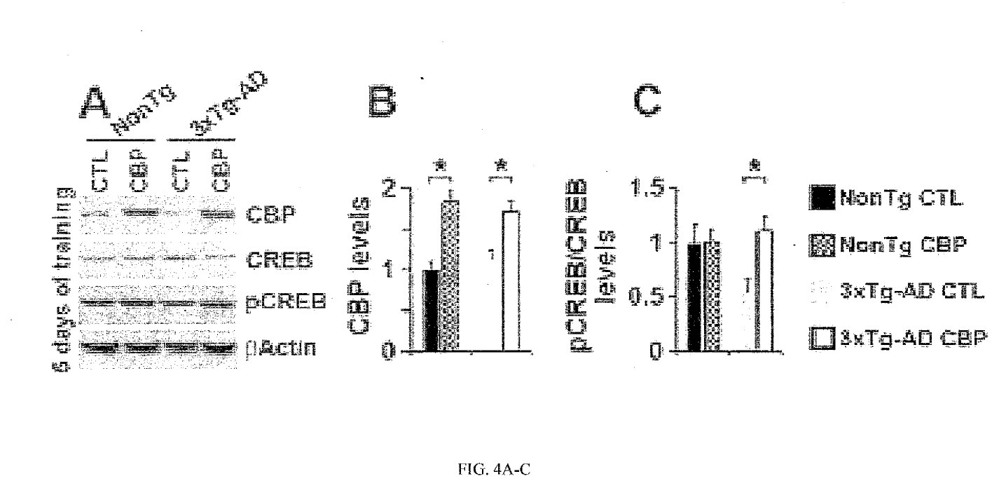

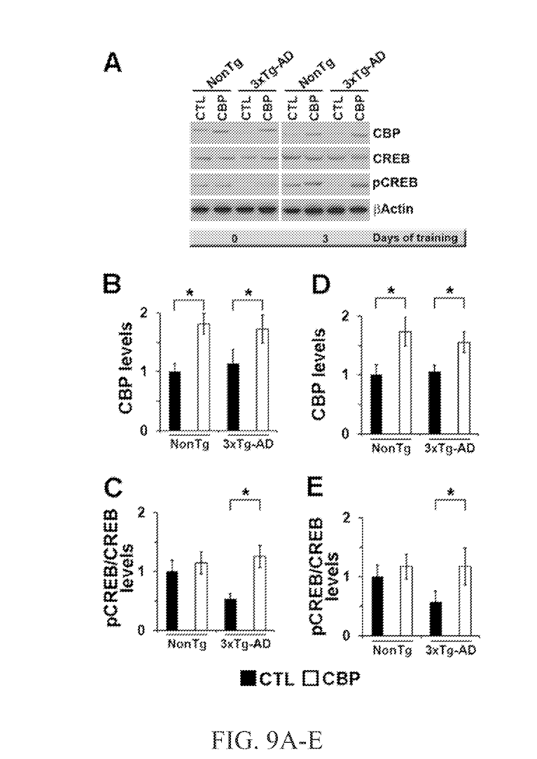

To elucidate the molecular basis underlying the CBP-mediated improvements in learning and memory, the inventor determined the consequences of CBP gene delivery on CREB function. The inventor first compared CBP levels between the hippocampi of CBP- and sham-injected mice at baseline (7 days post-injection but without water maze training), and after 3 and 5 days of training. CBP levels were significantly higher in the hippocampi of the CBP-injected mice at all three time-points, independent of the genotype (FIGS. 4A-B, and FIGS. 9A-B, D). Notably, at baseline, CBP levels were similar between sham-injected Non-Tg and 3×Tg-AD mice (FIGS. 9A-B), suggesting that CBP levels are not altered in the 3×Tg-AD mice. Further, CREB phosphorylation was not changed in the Non-Tg mice at any of the time-points analyzed (FIGS. 4A-C and 9A, 9C, 9E). In contrast, the inventor found that CBP overexpression restored CREB phosphorylation in the 3×Tg-AD mice at baseline and after 3 and 5 days of training (FIGS. 4A, 4C and FIGS. 9A, 9C, 9E. These data are consistent with the improvement in learning and memory in the 3×Tg-AD mice in a hippocampal-dependent task.

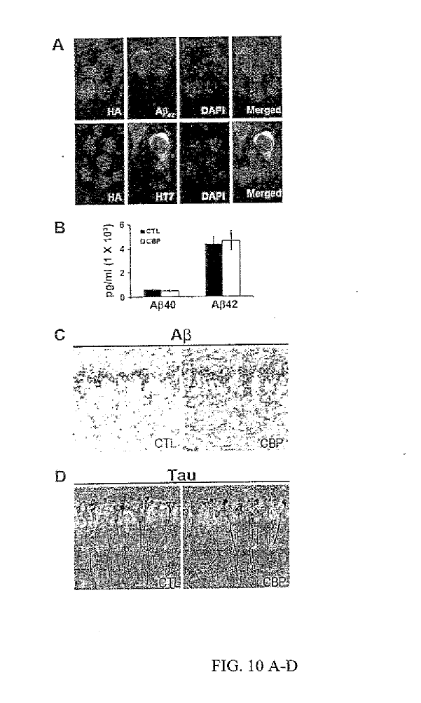

The onset of cognitive decline in the 3×Tg-AD mice is due to Aβ accumulation, which as the inventor shows here, is also responsible for the alteration in CREB phosphorylation (FIG. 6A-H). As the 3×Tg-AD mice age, cognitive decline becomes more severe and is also mediated by tau pathology ((Oddo et al., 2008). Notably, the lentivirus drove expression of CBP in Aβ₄₂- and tau-bearing neurons (FIG. 10A). Therefore, the inventor next asked whether the CBP-mediated improvement in behavior may be due to a decrease in Aβ and tau pathology. Sandwich ELISA measurements from protein extracted from the hippocampi of 3×Tg-AD mice showed that the levels of soluble Aβ40 and Aβ42 were similar between 3×Tg-AD mice receiving CBP and sham-injected 3×Tg-AD mice (FIG. 10B). Further, immunohistochemical analysis showed no changes in Aβ or tau immunoreactivity between these two groups of mice (FIGS. 10C-D).

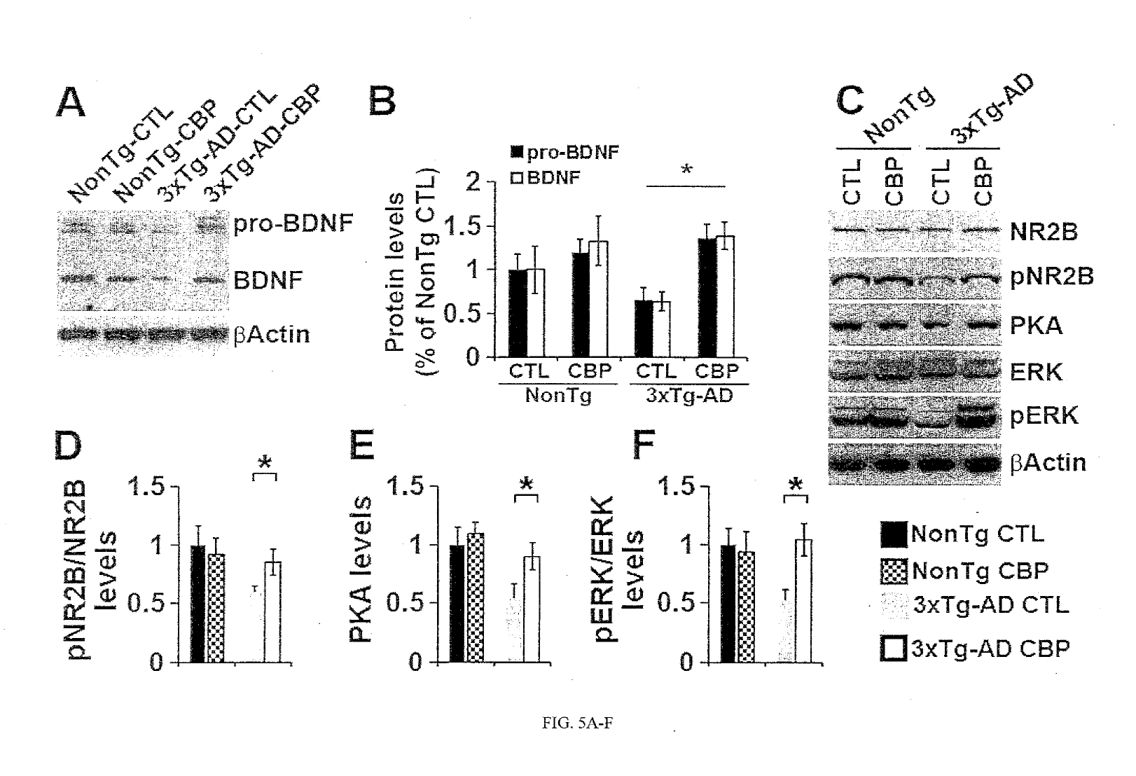

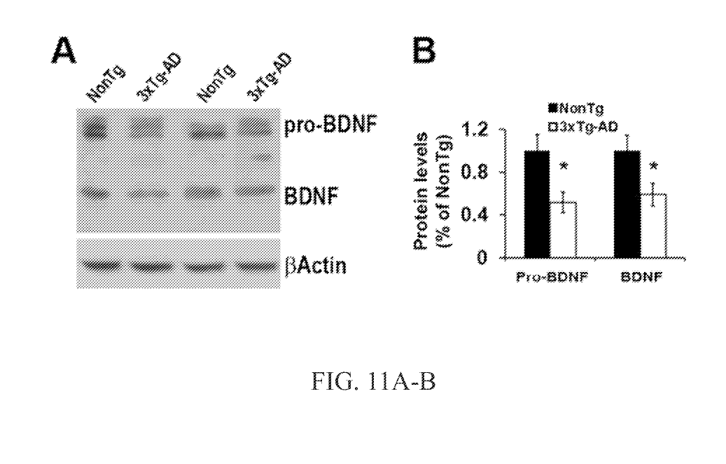

Taken together, the results presented so far indicate that restoration of CREB phosphorylation is sufficient to rescue learning and memory deficits without altering Aβ or tau levels. The role of CREB in cognition is well established and is thought that once activated, CREB facilitates the transcription of key proteins necessary for activity-dependent plasticity (Lee and Silva, 2009). One of these proteins is the brain-derived neurotrophic factor (BDNF), which facilitates synaptic plasticity and memory formation (Cowansage et al., 2010). Moreover, BDNF is proposed to play a role in the pathogenesis of AD, and its levels are reduced in AD brains (Hock et al., 2000; Connor et al., 1997). Thus, the inventor next sought to determine whether the improvement in learning and memory following CBP gene transfer may be linked to an increase in BDNF levels in the hippocampus. Toward this end, the inventor first measured the levels of BDNF in the hippocampi of 6-month-old 3×Tg-AD and Non-Tg mice (n=9/genotype) and found that BDNF levels were significantly decreased in the 3×Tg-AD mice (FIG. 11). These results are consistent with previous reports showing reductions in BDNF in AD brains and other animal models (Hock et al., 2000; Connor et al., 1997; Peng et al., 2009). The inventor next assessed how BDNF protein levels change in response to CBP gene transfer. Using Western blot analyses, the inventor found that in the Non-Tg mice receiving CBP, the levels of BDNF were higher, but not statistically significant, than sham-injected Non-Tg mice (FIGS. 5A-B). In contrast, the inventor found that BDNF protein levels were significantly higher in the hippocampi of the 3×Tg-AD mice compared to sham-injected 3×Tg-AD mice (FIGS. 5A-B). Notably, in the 3×Tg-AD mice, CBP gene delivery restored BDNF levels to Non-Tg levels (FIGS. 5A-B).

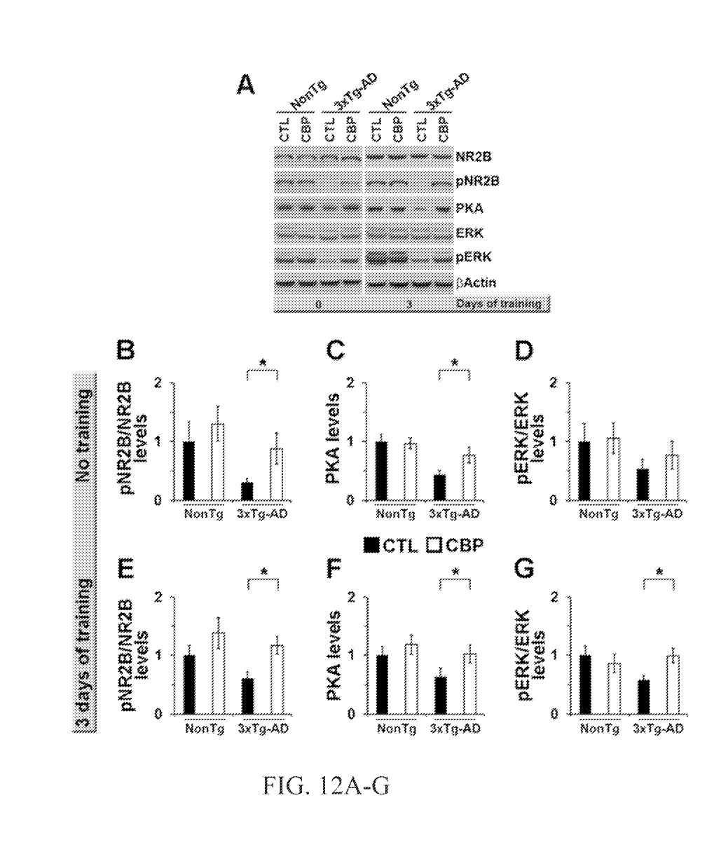

The data presented in FIGS. 2A-B indicate that NMDA signaling is decreased in 3×Tg-AD mice; notably, BDNF facilitates NMDA signaling and increases phosphorylation of NR2B (Xu et al., 2006). Thus, to determine whether the CBP-mediated increase in BDNF facilitates NMDA signaling, the inventor measured pNR2B levels at baseline and after 3 and 5 days of training. The inventor found that in the CBP-injected 3×Tg-AD mice, pNR2B levels were significantly higher at all three time-points, compared to sham-injected 3×Tg-AD mice (FIGS. 5C-D and FIGS. 12A-B, 12E). Previous reports indicate that an increase in pNR2B correlates with a higher NMDA signaling (Snyder et al., 2005). The inventor therefore measured PKA and ERK levels and found that PKA levels were significantly increased in the CBP-injected 3×Tg-AD mice compared to the sham-injected 3×Tg-AD mice, at all three time-points (FIGS. 5C, 5E and FIG. 12A, 12C, 12F). In contrast, the inventor found pERK levels were significantly higher in the CBP-injected 3×Tg-AD mice after 3 and 5 days of training but not at baseline (FIGS. 5C, 5F and FIGS. 12A-D, 12G). Finally, the inventor found that CBP injections did not alter NMDA signaling (FIGS. 5A-F and FIG. 12A-G). Considering the primary role of BDNF and NMDA signaling in activity-dependent synaptic plasticity, the data provided here suggest that some of the beneficial effect of CBP gene transfer on learning and memory may be linked to a BDNF-mediated increase in NMDA signaling.

### Example 3

### Discussion

Upon neuronal stimulation, CREB is normally phosphorylated at Ser133 and activated; this activation is necessary for memory formation and consolidation (Lee and Silva, 2009). Consistently, impairing CREB activation has detrimental effects on different forms of learning and memory, including spatial reference memory, a hippocampal-dependent form of memory that is highly impaired in people with AD (Lee and Silva, 2009). Here the inventor shows a ˜40% decrease in pCREB levels at baseline in the hippocampus of the 3×Tg-AD mice, which is consistent with previous reports (Ma et al., 2007). Most notably, the inventor further shows that the difference in pCREB levels between 3×Tg-AD and Non-Tg mice was greater upon neuronal stimulation; indeed, after 5 days of training, pCREB levels were ˜200% lower in the 3×Tg-AD mice compared to the Non-Tg mice. PKA and ERK are two kinases involved in CREB phosphorylation upon neuronal stimulation (Lee and Silva, 2009). Here the inventor provides compelling in vivo evidence showing that in the hippocampi of the 3×Tg-AD mice, Aβ accumulation is responsible for reduced pCREB levels. Considering the role of CREB during the formation of new memories, these data suggest some of the learning and memory deficits in the 3×Tg-AD mice may be mediated by deficits in CREB phosphorylation. These results, however, are not meant to imply that this is the only signaling pathway linking Aβ accumulation to cognitive decline; it is likely that alterations in other synaptic signaling could contribute to the learning and memory deficits. During neuronal stimulation, activation of NMDA receptors leads to PKA- and ERK-mediated activation and CREB phosphorylation, which is necessary for memory formation (Lee and Silva, 2009). Dysfunction in NMDA receptor signaling and trafficking has been reported in several animal models of AD (Snyder et al., 2005; Palop and Mucke, 2010; Li et al., 2009). Here the inventor shows that the phosphorylation of tyrosine 1472 of the NR2B subunit, which increases the activity of NMDA receptors (Snyder et al., 2005), was significantly decreased in the hippocampi of the 3×Tg-AD mice at baseline. The Aβ-induced alterations in NMDA/PKA/ERK signaling in the 3×Tg-AD mice may account for the attenuated response in CREB phosphorylation upon exposure to new learning stimuli.

Once phosphorylated at Ser133, CREB binds to the transcriptional co-activator CBP, which facilitates transcription by recruiting other components of the transcriptional machinery and by its intrinsic HAT activity (Vo and Goodman, 2001). Hence, CBP has been shown to enhance CREB-dependent transcription by directly acetylating CREB (Vo and Goodman, 2001). Notably, several memory-related signal transduction pathways known to be altered by Aβ accumulation are directly linked to CREB/CBP. Work by Saura and colleagues show that cognitive deficits in brain-specific presenilin (PS) 1 and 2 double-knockout mice are associated with a decrease in CBP levels and CREB/CBP mediated transcription (Saura et al., 2004). Consistently, in vitro data show that wild-type PS1 but not FAD mutant, facilitate CREB/CBP mediated transcription (Francis et al., 2006). These data contrast with a report showing that FAD mutations in the PS1 gene increase CREB/CBP mediated transcription (Marambaud et al., 2003). The results presented here support the hypothesis that PS loss of function and Aβ accumulation can synergistically cause cognitive impairments by interfering with CREB/CBP mediated transcription, as proposed by the Shen's group (Saura et al., 2004). Here the inventor shows that Aβ accumulation decreased CREB phosphorylation and more important, that increasing CBP expression in the hippocampi of adult 3×Tg-AD mice was sufficient to rescue learning and memory deficits without affecting Aβ or tau pathology. Although the 3×Tg-AD mice harbor a FAD mutation in the PS1 gene, the CREB deficits appear to be mediated by the accumulation of Aβ and not directly by the mutant PS1 protein. Indeed, the inventor shows that reducing Aβ levels by intrahippocampal injections of 6E10 rescued pCREB levels and injections of soluble Aβ was sufficient to induce pCREB deficits in wild-type mice.

These results show that the CBP-mediated improvement in learning and memory was linked to an increase of BDNF levels in the hippocampi. In turn, such an increase potentiates NMDA signaling, which may further facilitate CREB phosphorylation, creating a positive feed-forward loop. BDNF is a CREB target gene that plays a critical role in learning and memory, and there is a large body of literature showing BDNF dysfunction in AD. Indeed, Aβ accumulation decreases BDNF levels in vitro and in animal models of AD (Garzon and Fahnestock, 2007), and more important, BDNF levels are reduced in AD brains (Hock et al., 2000; Connor et al., 1997). Indeed, several therapeutic strategies designed at ameliorating AD pathology are linked to increased BDNF levels. For example, behavioral enrichment and physical exercise increase BDNF levels and ameliorate learning and memory deficits in animal models of AD (Fahnestock et al., 2010; Neeper et al., 1996). Recently, a pioneering work by the LaFerla's group has shown that neuronal stem cells improve learning and memory deficits in 3×Tg-AD mice by increasing BDNF levels without affecting Aβ and tau pathology (Blurton-Jones et al., 2009). Consistent with these findings, another report shows that BDNF administration improves learning and memory deficits in several animal models of AD, including a non-human primate, without affecting Aβ pathology (Nagahara et al., 2009). These reports and the data presented here are consistent with the view that BDNF deficits in AD are downstream of Aβ accumulation and the Aβ-induced dysfunction in CREB mediated transcription may account for the BDNF deficits. Although CBP levels were also increased in the hippocampi of Non-Tg mice, BDNF levels were not significantly higher in the CBP-injected compared to sham-injected Non-Tg mice, suggesting that the CREB-mediated BDNF transcription is tightly regulated under physiological conditions. Consistent with the inventor's hypothesis that NMDA/PKA/ERK signaling is restored because of an increase in BDNF levels, no changes were found in NMDA signaling were found in the CBP-injected Non-Tg mice.

In summary, these data indicate that cognitive dysfunction in AD can be restored without affecting Aβ or tau pathology and further support the use of gene transfer into adult brains as a potential therapeutic approach for AD and other related neurodegenerative disorders. Toward this end, it should be noted that CBP dysfunctions are also reported in other neurodegenerative disorders such as Huntington disease and Rubinstein-Taybi syndrome (Rouaux et al., 2004), suggesting that CBP-gene delivery may also have beneficial effects beyond AD.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.
U.S. Pat. No. 5,223,424
U.S. Pat. No. 5,626,850
U.S. Pat. No. 5,889,136
U.S. Pat. No. 5,929,237
U.S. Pat. No. 5,994,136
U.S. Pat. No. 6,165,782
U.S. Pat. No. 6,277,633
U.S. Pat. No. 6,319,703
U.S. Pat. No. 6,344,445
U.S. Pat. No. 6,428,953
U.S. Pat. No. 6,521,457
U.S. Pat. No. 6,924,144
U.S. Patent Pub. 20030147966
U.S. Patent Pub. 20030223938
U.S. Patent Pub. 20050143336
U.S. Provisional Application Ser. No. 60/661,680
Baichwal and Sugden, In: Gene Transfer, Kucherlapati (Ed.), NY, Plenum Press, 117-148, 1986.
Blurton-Jones et al., et al., Proc. Natl. Acad. Sci. USA, 106(32):13594-13599, 2009.
Caccamo et al., J. Biol. Chem., 285(17):13107-13120, 2010.
Cleary et al., Nat. Neurosci., 8(1):79-84, 2005.
Connor et al., Brain Res. Mol. Brain. Res., 49(1-2):71-81, 1997.
Cowansage et al., Curr. Molec. Pharmacol., 3(1):12-29, 2010.
Dickey et al., J. Neurosci., 23(12):5219-5226, 2003.
Espana et al., J. Neurosci., 30(28):9402-9410, 2010.
Fahnestock et al., Neurobiol Aging, 2010 (Epub ahead of Print)
Francis et al., Neuroreport., 17(9):917-921, 2006.
Garzon and Fahnestock, J. Neurosci., 27(10):2628-2635, 2007.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Goodman and Smolik, Genes and Develop., 14(13):1553-1577, 2000.
Hock et al., Archives Neurology, 57(6):846-851, 2000.
Kang et al., Nature, 325:733-736, 1987.
King & Turner, Exp Neural., 185(2):208-19, 2005.
Koo and Squazzo, J. Biol. Chem., 269(26):17386-17389, 1994.
Lanahan and Worley, Neurobiol. Learn Mem., 70(1-2):37-43, 1998.
Lee and Silva, Nature Rev., 10(2):126-140, 2009.
Li et al., Neuron, 62(6):788-801, 2009.
Ma et al., J. Neurochem., 103(4):1594-1607, 2007.
Marambaud et al., Cell, 114(5):635-645, 2003.
Nagahara et al., Nat. Med., 15(3):331-337, 2009.
Neeper et al., Brain Res., 726(1-2):49-56, 1996.
Nicolas and Rubenstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176, 1987.
Oddo et al., J. Neurosci., 28(47):12163-12175, 2008.
Oddo et al., Neuron, 39(3):409-421, 2003.
Oddo et al., Neuron, 43(3):321-332, 2004.
Palop and Mucke, Nat. Neurosci., 13(7):812-818, 2010.
Palop et al., J. Neurosci., 25(42):9686-9693, 2005.
Palop et al., Proc. Natl. Acad. Sci. USA, 100(16):9572-9577, 2003.
PCT Appln. WO 98/07408
Peng et al., J. Neurosci., 29(29):9321-9329, 2009.
Remington's Pharmaceutical Sciences, 18th Ed. Mack Printing Company, pp. 1289-1329, 1990.
Ridgeway, In: Vectors: A Survey of Molecular Cloning Vectors and Their Uses, Rodriguez et al. (Eds.), Stoneham: Butterworth, 467-492, 1988.
Rouaux et al., Biochem. Pharmacol., 68(6):1157-1164, 2004.
Saura et al., Neuron, 42(1):23-36, 2004.
Selkoe, Physiol. Rev., 81:741-766, 2001.
Smyth-Templeton et al., DNA Cell Biol., 21(12):857-867, 1997.
Snyder et al., Nat. Neurosci., 8(8):1051-1058, 2005.
Temin, In: Gene Transfer, Kucherlapati (Ed.), NY, Plenum Press, 149-188, 1986.
Tong et al., J. Biol. Chem., 276(20):17301-17306, 2001.
Vitolo et al., Proc. Nad. Acad. Sci. USA, 99(20):13217-13221, 2002.
Vo and Goodman, J. Biol. Chem., 276(17):13505-13508, 2001.
Wang et al., FASEB J., 24(10:4420-32, 2010.
Wong et al., Gene, 10:87-94, 1980.
Xu et al., Brain Res., 1121(1):22-34, 2006.

1. A method of increasing brain-derived neurotrophic factor (BDNF) in the brain of a subject comprising providing to said subject a CREB-binding protein (CBP).

2. The method of claim 1, wherein CBP is provided by administration of an expression vector to said subject.

3. The method of claim 1, wherein said administration comprises injection or infusion using stereotactic surgical techniques.

4. The method of claim 2, wherein said expression vector is a viral vector.

5. The method of claim 4, wherein said viral vector is neutrophic viral vector.

6. The method of claim 5, wherein said neurotrophic viral vector is a retroviral vector, a lentiviral vector, a herpesviral vector, an adenoviral vector or an adeno-associated viral vector.

7. The method of claim 2, wherein said expression vector is a non-viral vector.

8. The method of claim 7, wherein said non-viral vector is contained in a lipid delivery vehicle or nanoparticle.

9. The method of claim 8, wherein said lipid delivery vehicle is a liposome.

10. The method of claim 1, wherein said subject has been diagnosed with a neurodegenerative disease.

11. The method of claim 1, wherein said subject has not been diagnosed with a neurodegenerative disease.

12. The method of claim 1, wherein said subject has a familial history of neurodegenerative disease.

13. The method of claim 1, further comprising treating said subject with a second neurodegenerative disease therapy.

14. The method of claim 13, wherein said second neurodegenerative disease therapy is a cognitive therapy.

15. The method of claim 1, wherein providing comprises, daily, every other day, every third day, every fourth day, every fifth day, every sixth day or weekly administration.

16. A method of improving learning and/or reducing memory deficits in a subject comprising providing to said subject CREB-binding protein (CBP).

17-30. (canceled)

31. A method of treating a neurodegenerative disease in a subject comprising providing to said subject CREB-binding protein (CBP).

32-45. (canceled)

46. A pharmaceutical composition comprising an expression construct comprising a promoter active in neuronal cells operably connected to a nucleic acid segment coding for a CREB-binding protein (CBP) disposed in a pharmaceutically acceptable carrier, diluent or buffer.

47-50. (canceled)


SEQUENCE LISTING (2 sequences)

SEQ ID NO: 1 - DNA, 7507 bp, Homo sapiens
  CDS (182)..(7507)
gctgttgctgaggctgagatttggccgccgcctcccccacccggcctgcgccctccgcggcccggcccgcgctcctgcgctcgctcctcgctggctcgcctgctcgcagccgccggcccgacccccgtccgggccgcgtcgcgccgcccgcgctcagggctgtttccgcgagcaggtgaagatggccgagaacttgctggacggaccgcccaaccccaaacgagccaaactcagctcgcccggcttctccgcgaatgacaacacagattttggatcattgtttgacttggaaaatgaccttcctgatgagctgatccccaatggagaattaagccttttaaacagtgggaaccttgttccagatgctgcgtccaaacataaacaactgtcagagcttcttagaggaggcagcggctctagcatcaacccagggataggcaatgtgagtgccagcagccctgtgcaacagggccttggtggccaggctcaggggcagccgaacagtacaaacatggccagcttaggtgccatgggcaagagccctctgaaccaaggagactcatcaacacccaacctgcccaaacaggcagccagcacctctgggcccactccccctgcctcccaagcactgaatccacaagcacaaaagcaagtagggctggtgaccagtagtcctgccacatcacagactggacctgggatctgcatgaatgctaacttcaaccagacccacccaggccttctcaatagtaactctggccatagcttaatgaatcaggctcaacaagggcaagctcaagtcatgaatggatctcttggggctgctggaagaggaaggggagctggaatgccctaccctgctccagccatgcagggggccacaagcagtgtgctggcggagaccttgacacaggtttccccacaaatggctggccatgctggactaaatacagcacaggcaggaggcatgaccaagatgggaatgactggtaccacaagtccatttggacaaccctttagtcaaactggagggcagcagatgggagccactggagtgaacccccagttagccagcaaacagagcatggtcaatagtttacctgcttttcctacagatatcaagaatacttcagtcaccactgtgccaaatatgtcccagttgcaaacatcagtgggaattgtacccacacaagcaattgcaacaggccccacagcagaccctgaaaaacgcaaactgatacagcagcagctggttctactgcttcatgcccacaaatgtcagagacgagagcaagcaaatggagaggttcgagcctgttctctcccacactgtcgaaccatgaaaaacgttttgaatcacatgacacattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctgggatctccagctagtggaattcaaaacacaattggttctgttggtgcagggcaacagaatgccacttccttaagtaacccaaatcccatagaccccagttccatgcagcgggcctatgctgctctaggactcccctacatgaaccagcctcagacgcagctgcagcctcaggttcctggccagcaaccagcacagcctccagcccaccagcagatgaggactctcaatgccctaggaaacaaccccatgagtatcccagcaggaggaataacaacagatcaacagccaccaaacttgatttcagaatcagctcttccaacttccttgggggctaccaatccactgatgaatgatggttcaaactctggtaacattggaagcctcagcacgatacctacagcagcgcctccttccagcactggtgttcgaaaaggctggcatgaacatgtgactcaggacctacggagtcatctagtccataaactcgttcaagccatcttcccaactccagaccctgcagctctgaaagatcgccgcatggagaacctggttgcctatgctaagaaagtggagggagacatgtatgagtctgctaatagcagggatgaatactatcatttattagcagagaaaatctataaaatacaaaaagaactagaagaaaagcggaggtcacgtttacataagcaaggcatcctgggtaaccagccagctttaccagcttctggggctcagccccctgtgattccaccagcccagtctgtaagacctccaaatgggcccctgcctttgccagtgaatcgcatgcaggtttctcaagggatgaattcatttaacccaatgtccctgggaaacgtccagttgccacaggcacccatgggacctcgtgcagcctcccctatgaaccactctgtgcagatgaacagcatggcctcagttccgggtatggccatttctccttcacggatgcctcagcctccaaatatgatgggcactcatgccaacaacattatggcccaggcacctactcagaaccagtttctgccacagaaccagtttccatcatccagtggggcaatgagtgtgaacagtgtgggcatggggcaaccagcagcccaggcaggtgtttcacagggtcaggtacctggagctgctctccctaaccctctgaacatgctggcaccccaggccagccagctgccttgcccaccagtgacacagtcaccattgcacccgactccacctcctgcttccacagctgctggcatgccctctctccaacatccaacggcaccaggaatgacccctcctcagccagcagctcccactcagccatctactcctgtgtcatctgggcagactcctaccccaactcctggctcagtgcccagcgctgcccaaacacagagtacccctacagtccaggcagcagcacaggctcaggtgactccacagcctcagaccccagtgcagccaccatctgtggctactcctcagtcatcacagcagcaaccaacgcctgtgcatactcagcctcctggcacaccgctttctcaggcagcagccagcattgataatagagtccctactccctcctctgtgaccagtgctgaaaccagttcccagcagccaggacccgatgtgcccatgctggaaatgaagacagaggtgcagacagatgatgctgagcctgaacctactgaatccaagggggaacctcggtctgagatgatggaagaggatttacaaggttcttcccaagtaaaagaagagacagatacgacagagcagaagtcagagccaatggaagtagaagaaaagaaacctgaagtaaaagtggaagctaaagaggaagaagagaacagttcgaacgacacagcctcacaatcaacatctccttcccagccacgcaaaaaaatctttaaacccgaggagctacgccaggcacttatgccaactctagaagcactctatcgacaggacccagagtctttgccttttcgtcagcctgtagatcctcagctcctaggaatcccagattattttgatatagtgaagaatcctatggacctttctaccatcaaacgaaagctggacacagggcaatatcaagaaccctggcagtatgtggatgatgtctggcttatgttcaacaatgcgtggctatataatcgtaaaacgtcccgtgtatataaattttgcagtaaacttgcagaggtctttgaacaagaaattgaccctgtcatgcagtctcttggatattgctgtggacgaaagtatgagttctccccacagactttgtgctgttacggaaagcagctgtgtacaattcctcgtgatgcagcctactacagctatcagaataggtatcatttctgtgagaagtgtttcacagagatccagggcgagaatgtgaccctgggtgacgacccttcccaacctcagacgacaatttccaaggatcaatttgaaaagaagaaaaatgataccttagatcctgaaccttttgttgactgcaaagagtgtggccggaagatgcatcagatttgtgttctacactatgacatcatttggccttcaggttttgtgtgtgacaactgtttgaagaaaactggcagacctcggaaagaaaacaaattcagtgctaagaggctgcagaccacacgattgggaaaccacttagaagacagagtgaataagtttttgcggcgccagaatcaccctgaagctggggaggtttttgtcagagtggtggccagctcagacaagactgtggaggtcaagccgggaatgaagtcaaggtttgtggattctggagagatgtcggaatctttcccatatcgtaccaaagcactctttgcttttgaggagatcgatggagtcgatgtgtgcttttttgggatgcatgtgcaagaatacggctctgattgccccccaccaaatacaaggcgtgtatacatatcttatctggacagtattcatttcttccggccccgctgcctccggacagctgtttaccatgagatcctcatcggatatctcgagtatgtgaagaaattggggtatgtgacaggacatatttgggcctgtcccccaagtgaaggagatgactatatctttcattgccacccccctgaccagaaaatccccaaaccaaaacgactacaggagtggtacaagaagatgctggacaaggcgtttgcagagaggatcattaacgactataaggacatcttcaaacaagcgaacgaagacaggctcacgagtgccaaggagttgccctattttgaaggagatttctggcctaatgtgttggaagaaagcattaaggaactagaacaagaagaagaagaaaggaaaaaagaagagagtactgcagcgagtgagactcctgagggcagtcagggtgacagcaaaaatgcgaagaaaaagaacaacaagaagaccaacaaaaacaaaagcagcattagccgcgccaacaagaagaagcccagcatgcccaatgtttccaacgacctgtcgcagaagctgtatgccaccatggagaagcacaaggaggtattctttgtgattcatctgcatgctgggcctgttatcagcactcagccccccatcgtggaccctgatcctctgcttagctgtgacctcatggatgggcgagatgccttcctcaccctggccagagacaagcactgggaattctcttccttacgccgctccaaatggtccactctgtgcatgctggtggagctgcacacacagggccaggaccgctttgtttatacctgcaatgagtgcaaacaccatgtggaaacacgctggcactgcactgtgtgtgaggactatgacctttgtatcaattgctacaacacaaagagccacacccataagatggtgaagtgggggctaggcctagatgatgagggcagcagtcagggtgagccacagtccaagagcccccaggaatcccggcgtctcagcatccagcgctgcatccagtccctggtgcatgcctgccagtgtcgcaatgccaactgctcactgccgtcttgccagaagatgaagcgagtcgtgcagcacaccaagggctgcaagcgcaagactaatggaggatgcccagtgtgcaagcagctcattgctctttgctgctaccacgccaaacactgccaagaaaataaatgccctgtgcccttctgcctcaacatcaaacataagctccgccagcagcagatccagcatcgcctgcagcaggctcagctcatgcgccggcgaatggcaaccatgaacacccgcaatgtgcctcagcagagtttgccttctcctacctcagcaccacccgggactcctacacagcagcccagcacaccccaaacaccacagcccccagcccagcctcagccttcacctgttaacatgtcaccagctggcttccctaatgtagcccggactcagcccccaacaatagtgtctgctgggaagcctaccaaccaggtgccagctcccccaccccctgcccagcccccacctgcagcagtagaagcagcccggcaaattgaacgtgaggcccagcagcagcagcacctataccgagcaaacatcaacaatggcatgcccccaggacgtgcaggtatggggaccccaggaagccaaatgactcctgtgggcctgaatgtgccccgtcccaaccaagtcagtgggcctgtcatgtctagtatgccacctgggcagtggcagcaggcacccatccctcagcagcagccgatgccaggcatgcccaggcctgtaatgtccatgcaggcccaggcagcagtggctgggccacggatgcccaatgtgcagccaccaaggagcatctcgccaagtgccctgcaagacctgctacggaccctaaagtcacccagctctcctcagcagcagcagcaggtgctgaacatccttaaatcaaacccacagctaatggcagctttcatcaaacagcgcacagccaagtatgtggccaatcagcctggcatgcagccccagcccggacttcaatcccagcctggtatgcagccccagcctggcatgcaccagcagcctagtttgcaaaacctgaacgcaatgcaagctggtgtgccacggcctggtgtgcctccaccacaaccagcaatgggaggcctgaatccccagggacaagctctgaacatcatgaacccaggacacaaccccaacatgacaaacatgaatccacagtaccgagaaatggtgaggagacagctgctacagcaccagcagcagcagcagcaacagcagcagcagcagcagcaacaacaaaatagtgccagcttggccgggggcatggcgggacacagccagttccagcagccacaaggacctggaggttatgccccagccatgcagcagcaacgcatgcaacagcacctccccatccagggcagctccatgggccagatggctgctccaatgggacaacttggccagatggggcagcctgggctaggggcagacagcacccctaatatccagcaggccctgcagcaacggattctgcagcagcagcagatgaagcaacaaattgggtcaccaggccagccgaaccccatgagcccccagcagcacatgctctcaggacagccacaggcctcacatctccctggccagcagatcgccacatcccttagtaaccaggtgcgatctccagcccctgtgcagtctccacggccccaatcccaacctccacattccagcccgtcaccacggatacaaccccagccttcaccacaccatgtttcaccccagactggttcccctcaccctggactcgcagtcaccatggccagctccatggatcagggacacctggggaaccctgaacagagtgcaatgctcccccagctgaatacccccaacaggagcgcactgtccagtgaactgtccctggttggtgataccacgggagacacactagaaaagtttgtggagggtttgtag

SEQ ID NO: 2 - protein, 2441 aa, Homo sapiens
MAENLLDGPPNPKRAKLSSPGFSANDNTDFGSLFDLENDLPDELIPNGELSLLNSGNLVPDAASKHKQLSELLRGGSGSSINPGIGNVSASSPVQQGLGGQAQGQPNSTNMASLGAMGKSPLNQGDSSTPNLPKQAASTSGPTPPASQALNPQAQKQVGLVTSSPATSQTGPGICMNANFNQTHPGLLNSNSGHSLMNQAQQGQAQVMNGSLGAAGRGRGAGMPYPAPAMQGATSSVLAETLTQVSPQMAGHAGLNTAQAGGMTKMGMTGTTSPFGQPFSQTGGQQMGATGVNPQLASKQSMVNSLPAFPTDIKNTSVTTVPNMSQLQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGAGQQNATSLSNPNPIDPSSMQRAYAALGLPYMNQPQTQLQPQVPGQQPAQPPAHQQMRTLNALGNNPMSIPAGGITTDQQPPNLISESALPTSLGATNPLMNDGSNSGNIGSLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPASGAQPPVIPPAQSVRPPNGPLPLPVNRMQVSQGMNSFNPMSLGNVQLPQAPMGPRAASPMNHSVQMNSMASVPGMAISPSRMPQPPNMMGTHANNIMAQAPTQNQFLPQNQFPSSSGAMSVNSVGMGQPAAQAGVSQGQVPGAALPNPLNMLAPQASQLPCPPVTQSPLHPTPPPASTAAGMPSLQHPTAPGMTPPQPAAPTQPSTPVSSGQTPTPTPGSVPSAAQTQSTPTVQAAAQAQVTPQPQTPVQPPSVATPQSSQQQPTPVHTQPPGTPLSQAAASIDNRVPTPSSVTSAETSSQQPGPDVPMLEMKTEVQTDDAEPEPTESKGEPRSEMMEEDLQGSSQVKEETDTTEQKSEPMEVEEKKPEVKVEAKEEEENSSNDTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIINDYKDIFKQANEDRLTSAKELPYFEGDFWPNVLEESIKELEQEEEERKKEESTAASETPEGSQGDSKNAKKKNNKKTNKNKSSISRANKKKPSMPNVSNDLSQKLYATMEKHKEVFFVIHLHAGPVISTQPPIVDPDPLLSCDLMDGRDAFLTLARDKHWEFSSLRRSKWSTLCMLVELHTQGQDRFVYTCNECKHHVETRWHCTVCEDYDLCINCYNTKSHTHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVNMSPAGFPNVARTQPPTIVSAGKPTNQVPAPPPPAQPPPAAVEAARQIEREAQQQQHLYRANINNGMPPGRAGMGTPGSQMTPVGLNVPRPNQVSGPVMSSMPPGQWQQAPIPQQQPMPGMPRPVMSMQAQAAVAGPRMPNVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPPQPAMGGLNPQGQALNIMNPGHNPNMTNMNPQYREMVRRQLLQHQQQQQQQQQQQQQQQNSASLAGGMAGHSQFQQPQGPGGYAPAMQQQRMQQHLPIQGSSMGQMAAPMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSMDQGHLGNPEQSAMLPQLNTPNRSALSSELSLVGDTTGDTLEKFVEGL